(19) United States
(12) Patent Application Publication
(10) Pub. No.: US 2024/0409594 A1
(43) Pub. Date: Dec. 12, 2024

(54) PHOTOACTIVATABLE ION CHANNEL MODULATOR

(71) Applicants: INSTITUT NATIONAL DE LA SANTÉ ET DE LA RECHERCHE MÉDICALE, Paris (FR); NANTES UNIVERSITÉ, Nantes (FR); CENTRE NATIONAL DE LA RECHERCHE SCIENTIFIQUE, Paris (FR)

(72) Inventors: Michel DE WAARD, Nantes (FR); Jérôme MONTNACH, Nantes (FR)

(21) Appl. No.: 18/702,506

(22) PCT Filed: Oct. 18, 2022
(86) PCT No.: PCT/IB2022/059988

§ 371 Date: Apr. 18, 2024

(30) Foreign Application Priority Data
Oct. 18, 2021 (WO) PCT/IB2021/000737

(51) Int. Cl. C07K 14/435; A61K 38/00

(52) U.S. Cl. CPC C07K 14/43518; C07K 14/43522; A61K 38/00; C07K 2319/00

(57) ABSTRACT

Despite the presence of multiple disulphide bridges and the complicated tertiary structure of the venom peptide, the inventors in the present application showed that caging strategy involving covalent attachment of a photolabile protecting group on the lateral chain of a key residue for venom peptide activity causes steric clashes which are important enough to reduce the ion channel modulation efficacy. The inventors showed that the photoactivatable venom peptide which presents a shift of at least 100-fold of the dose-response value of normalized ion channel current in comparison to wild-type venom peptide is required to be effective under physiological conditions. The inventors showed for the first time that the chemical and photosensitive properties conferred to toxins allowed to probe the role of ion channel function in vivo with high spatial resolution making its therapeutic use possible. As further shown by the inventors, the technique can be generalized to toxins possessing more or less ion channel selectivity and is applicable to both inhibitors and activators. The present disclosure relates to a photoactivatable ion channel modulator, in particular for use in the treatment of an ion channel-related disease wherein said photoactivatable ion channel modulator is a disulphide-rich venom peptide comprising a photolabile protecting group.

### FIELD OF THE INVENTION

The present disclosure relates to a photoactivatable ion channel modulator, in particular for use in the treatment of an ion channel-related disease wherein said photoactivatable ion channel modulator is a disulphide-rich venom peptide comprising a photolabile protecting group.

### BACKGROUND OF THE INVENTION

Ion channels are pore-forming transmembrane proteins that allow the regulated flow of cations or anions across membranes. Due to their important biological role in many cell types, ion channels constitute drug targets for the treatment of diseases such as type-2 diabetes, hypertension, epilepsy, cardiac arrhythmia, and anxiety, and many are part of the classical drugs on the WHO's list of essential medicines (https://list.essentialmeds.org). Ion channels have long been regarded as difficult drug targets due to the challenge to achieve subtype selectivity and because they represent complex protein structures embedded in the plasma membrane. Biological compounds, such as peptides found in animal venoms, have demonstrated their usefulness in reaching high selectivity and affinity towards their targets owing to their larger chemical surface than small organic compounds. As such, they represent exquisite high affinity and selective tools for the pharmacological control of ion channels and of cellular excitability (Kalia, J. et al. Journal of molecular biology 427, 158-175 (2015)) and played a vital role in the deorphanization and classification of ion channels (Gonoi, T., Sherman, S. J. & Catterall, W. A. The Journal of neuroscience: the official journal of the Society for Neuroscience 5, 2559-2564 (1985)). While toxins originate from a wide variety of venomous species, they have in common a compact 3D structure, often largely imposed by internal disulphide bridges that promote the formation, organization and stabilization of secondary structures (Mouhat, S., et al. Biochem J 378, 717-726, (2004)). Mechanistically, peptides targeting ion channel can inhibit the pore or modify the gating process to alter channel activation or inactivation properties and thereby act as inhibitors or activators (Ahern, C. A., et al. J Gen Physiol 147, 1-24, (2016)). Another interesting feature is the diversity in selectivity encountered so far. Some venom peptides such as μ-conotoxin-GVIA (N-type Ca_{V} channel⁵), BeKm1 (hERG channel⁶) or μ-conotoxin KIIIA (Na_{V}1.2⁷) are selective for a particular target. Others bind to a virtually entire class of ion channels (i.e. AaHII and Na_{V} channels (Clairfeuille, T. et al. Science 363, (2019)). Hence, venom peptides appear as the most promising class of compounds to selectively control the activity of excitable cells.

When it comes to provide a light-mediated control of cell excitability, the technology that has been developed the furthest is optogenetics as witnessed by the substantial amount of literature on the topic (Joshi, J., Rubart, M. & Zhu, W. Frontiers in Bioengineering and Biotechnology 7 (2020); Entcheva, E. & Kay, M. W. Nat Rev Cardiol, (2020)). Undoubtedly, optogenetics have transformed many areas of biological research demonstrating the usefulness for a precise control of biological functions thanks to the unsurpassed spatial and temporal resolution of focused light. However, this technology does suffer from several drawbacks that are challenging to overcome for therapeutic applications, except on rare occasions such as recently shown for vision purpose (Tochitsky, I., et al. Chemical reviews 118, 10748-10773, (2018)). Optogenetics is largely based on the use of genetically-encoded material that needs to be supplied externally for invasive modification of the cell genome and proteome.

In contrast, photopharmacology does not require any modification of the cell proteome or genetic background and responds more favourably to the exigences of regulatory agencies for therapeutic development. Similarly to optogenetics, photoactivatable caged compounds are powerful tools for modulating the function of native proteins with high spatiotemporal resolution. This concept has shown promise in animal models for vision restoration (Tochitsky, I., et al. Chemical reviews 118, 10748-10773, (2018)) and pain management (Mourot, A. et al. Nat Methods 9, 396-402, (2012)). Although uncaging of chemicals for ligand-gated channels enabled seminal optopharmacology studies informing on their function and subcellular location in complex biological environments (Tazerart, S., et al. Nature communications 11, 4276, (2020); Matsuzaki, et al. Nature chemical biology 6, 255-257, (2010); Ellis-Davies, G. C. Nat Methods 4, 619-628, (2007); Dembitskaya, Y., Wu, Y. W. & Semyanov, A. The Journal of neuroscience: the official journal of the Society for Neuroscience 40, 4266-4276, (2020)), caged ligands targeting more specifically voltage-gated ion channels remain rare (Elleman, A. V. et al. Nature communications 12, 4171, (2021)), in part due to the lack of selective compounds.

A similar approach has been used in the past on Cys residues to control region-selective disulphide bonding of oxytocin, α-conotoxin IMI and human insulin (Karas, J. A. et al. Chemistry 20, 9549-9552, (2014)), but never to endow the pharmacological action of structurally complex toxins with a photosensitivity feature.

Thus, it remains a need to develop photoactivatable ion channel modulator based on venom peptides which present complex structure to allow spatial and temporal control of ion channel activity.

### SUMMARY OF THE INVENTION

Despite the presence of multiple disulphide bridges and the complicated tertiary structure of the venom peptide, the inventors in the present application showed that caging strategy involving covalent attachment of a photolabile protecting group on the lateral chain of a key residue for venom peptide activity causes steric clashes which are important enough to reduce the ion channel modulation efficacy. The inventors showed that the photoactivatable venom peptide which presents a shift of at least 100-fold of the dose-response value of normalized ion channel current in comparison to wild-type venom peptide is required to be effective under physiological conditions. The inventors showed for the first time that the chemical and photosensitive properties conferred to toxins allowed to probe the role of ion channel function in vivo with high spatial resolution making its therapeutic use possible. As further shown by the inventors, the technique can be generalized to toxins possessing more or less ion channel selectivity and is applicable to both inhibitors and activators.

The present disclosure relates to a photoactivatable ion channel modulator for use as medicament, in particular for use in the treatment of an ion channel-related disease wherein said photoactivatable ion channel modulator is a disulphide-rich venom peptide comprising a photolabile protecting group. Preferably, said photolabile protecting group molecule is selected from the group consisting of: nitrobenzyl-based photolabile protecting group, carbonyl-based photolabile protecting group and benzyl-based photolabile protecting group, more preferably nitrobenzyl-based photolabile such as 4,5-dimethoxy-2-nitrobenzyl (NVOC) group. In a preferred embodiment, a lysine, a tyrosine, serine, glycine or cysteine, preferably a lysine of said venom peptide is bound to said photolabile protecting group.

In a particular embodiment, said photoactivatable ion channel modulator is a photoactivatable voltage-gated sodium channel inhibitor, preferably a Huwentoxin-IV comprising or consisting of amino acid sequence selected from SEQ ID NO: 2 to 8 or functional variant thereof and wherein lysine at position 32 is bound to a photolabile protecting group, more preferably for use in the treatment of ion channel-related disease caused by abnormal cell membrane excitability, preferably selected from the group consisting of: epilepsy, Dravet syndrome, convulsion, cardiac arrythmia, pain, erythromelalgia, lumbosacral radiculopathy and trigeminal neuralgia.

In another particular embodiment, said photoactivatable ion channel modulator is a photoactivatable voltage-gated ion channel activator, preferably for use in the treatment of ion channel-related disease such as a neuromuscular junction disorder, preferably selected from the group consisting of: myasthenia gravis, autoimmune neuromyotonia or Lambert-Eaton syndrome, or congenital and familial neuromuscular disorders such as congenital myasthenia gravis syndrome.

In another aspect, the present disclosure relates to a non-therapeutic use of a photoactivatable ion channel modulator as defined above for modulating activity of ion channel of a cell in a tissue, wherein said venom peptide is activated by irradiating said tissue at appropriate light wavelength, preferably wherein said photolabile protecting group is nitrobenzyl-based photolabile protecting group and said venom peptide is activated by irradiating said tissue at wavelength above 340 nm. In a preferred embodiment, the present disclosure relates to a non-therapeutic use of a photoactivatable ion channel modulator as defined above for reducing soft-tissue feature, preferably wherein said photoactivatable ion channel modulator is photoactivatable voltage-gated sodium channel inhibitor such as Huwentoxin-IV comprising or consisting of amino acid sequence selected from SEQ ID NO: 2 to 8 or functional variant thereof and wherein lysine at position 32 is bound to a photolabile protecting group.

The present disclosure also relates to a disulphide-rich venom peptide comprising a photolabile protecting group wherein said venom peptide is a photoactivatable voltage-gated sodium channel inhibitor, preferably wherein said venom peptide is Huwentoxin-IV comprising or consisting of an amino sequence selected from SEQ ID NO: 2 to 8 or functional variant thereof and wherein a lysine at position 32 is bound to a photolabile protecting group, a disulphide-rich venom peptide comprising a photolabile protecting group wherein said venom peptide is a photoactivatable voltage-gated sodium activator, preferably wherein said venom peptide is Charybdotoxin comprising or consisting of SEQ ID NO: 10 or functional variant thereof and wherein a lysine at position 27, an asparagine at position 30 and/or tyrosine at position 36 is bound to a photolabile protecting group or a disulphide-rich venom peptide comprising a photolabile protecting group wherein said venom peptide is a photoactivatable voltage-gated potassium activator, preferably wherein said venom peptide is AahII comprising or consisting of SEQ ID NO: 12 or functional variant thereof and wherein an arginine residue is replaced by a lysine residue at position 62, itself bound to a photolabile protecting group. In a preferred embodiment, said photolabile protecting group molecule is selected from the group consisting of: nitrobenzyl-based photolabile protecting group, carbonyl-based photolabile protecting group and benzyl-based photolabile protecting group, preferably nitrobenzyl-based photolabile protecting group such as 4,5-Dimethoxy-2-nitrobenzyl (NVOC) group.

### BRIEF DESCRIPTION OF THE FIGURES

FIG. 1: photoactivatable HwTxIV-Nvoc analogue to modulate NaV channels: (a) Structure of caged HwTxIV-Nvoc analog with K32 and Nvoc group. (b) Average dose-response curves for Na_{V}1.1, Na_{V}1.2 and Na_{V}1.6 currents by non-caged HwTxIV analog. The data were fitted according to a Hill equation study (Na_{V}1.1: 18.9±1.2 nM, n=3-10 for HwTxIV analog versus 50.7±1.2 nM, n=3-6 for HwTxIV; Na_{V}1.2: 11.9±1.1 nM, n=4-9 for HwTxIV analog versus 8.3±1.2 nM, n=6-11 for HwTxIV; Na_{V}1.6: 19.5±1.3 nM, n=6-10 for HwTxIV analog versus 154.3±1.2 nM, n=3-7 for HwTxIV). (c) Average dose-response curves for hNa_{V}1.7 current inhibition by HwTxIV (black), HwTxIV-K32N mutant (grey). The data were fitted according to a Hill equation (IC₅₀>1 μM, n=4-7 for HwTxIV-N32 versus IC₅₀=9.9 nM±0.9 nM, n=5-6 for HwTxIV).

FIG. 2: Caged HwTxIV analogue drastically reduce affinity of HwTxIV analogue for NaV channels. (a) i) Representative recording of Na_{V}1.6 currents elicited at 10 mV from a holding potential of −100 mV illustrating the extent of current block by different concentrations of non-caged HwTxIV analog (black) and caged HwTxIV-Nvoc (grey). ii) Average dose-response curves for Na_{V}1.6 current inhibitions by non-caged HwTxIV analogue (black) and caged HwTxIV-Nvoc (grey). The data were fitted according to a Hill equation (non-caged HwTxIV analog IC₅₀=22.4 nM (n=5) and caged HwTxIV-Nvoc analogue IC₅₀=6539 nM (n=7)). Scales: 2 ms, 1 nA. Note that on hNa_{V}1.6, high concentrations of caged HwTxIV-Nvoc produce a slowing of channel inactivation due to binding on a low affinity site absent on hNa_{V}1.2. (b) i) Representative recording of hNa_{V}1.1 current elicited at 10 mV from a holding potential of −100 mV illustrating the extent of current block by different concentrations of non-caged HwTxIV analog (black) and caged HwTxIV-Nvoc (grey). ii) Average dose-response curves for hNa_{V}1.1 current inhibition by non-caged HwTxIV analog (black) and caged HwTxIV-Nvoc (grey). The data were fitted according to a Hill equation (non-caged HwTxIV analog IC₅₀=3.60 nM (n=12); caged HwTxIV-Nvoc analog IC₅₀=2794 nM (n=8) (776-fold reduction in IC₅₀). Scales: 2 ms, 1 nA. (c) i) Representative recording of hNa_{V}1.2 current elicited at 10 mV from a holding potential of ˜100 mV illustrating the extent of current block by different concentrations of non-caged HwTxIV analog (black) and caged HwTxIV-Nvoc (grey). Scales: 2 ms, 1 nA. ii) Average dose-response curves for hNa_{V}1.2 current inhibitions by non-caged HwTxIV analog (black) and caged HwTxIV-Nvoc (grey). The data were fitted according to a Hill equation (non-caged HwTxIV analog IC₅₀=3.23 nM (n=11); caged HwTxIV-Nvoc analog IC₅₀=5141 nM (n=6) (1592-fold reduction in IC₅₀).

FIG. 3: Physico-chemical and electrophysiogical properties of uncaged HwTxIV-Nvoc analogue (a) Top: Analytical RP-HPLC profiles of caged HwTxIV-Nvoc analog with different durations of illuminations (365 nm, 45 mW/cm²) demonstrating time-dependent control of uncaging. Bottom: Uncaged/Caged ratio of HPLC chromatogram peaks area versus irradiation time at 365 nm (45 mW/cm²). (b) Top: Analytical RP-HPLC profiles of caged HwTxIV-Nvoc analog with different intensity of illuminations (365 nm, 5 min) demonstrating intensity-dependent control of uncaging. Bottom: Uncaged/Caged ratio of HPLC chromatogram peaks area versus irradiation power at 365 nm (3 min). (c) Analytical RP-HPLC profiles of non-caged HwTxIV analog (top), 50:50 ratio of non-caged HwTxIV analog with partially uncaged HwTxIV-Nvoc analog (middle) and of caged HwTxIV-Nvoc analog (bottom). (d) Mass analyses of non-caged and uncaged HwTxIV analog by LC-ESI QTOF ([M+6H]6+ values). (e) Representative recording of hNa_{V}1.6 currents elicited at 10 mV from a holding potential of −100 mV illustrating the extent of current block by different concentrations of non-caged HwTxIV analog (black) and uncaged HwTxIV-Nvoc (purple). Scales: 2 ms, 1 nA. (f) Average dose-response curves for hNa_{V}1.6 current inhibitions by non-caged HwTxIV analog (black) and uncaged HwTxIV-Nvoc (purple). The data were fitted according to a Hill equation (non-caged HwTxIV analog IC50=22.4 nM (n=5) and uncaged HwTxIV-Nvoc analog IC₅₀=15.2 nM (n=6)).

FIG. 4: induced uncaging of toxins modulation ion channels properties. (a-c) Light-induced inhibition of hNa_{V}1.6 current by uncaged HwTxIV-Nvoc analog (100 nM). (a) Representative recording of hNa_{V}1.6 current in control (dark), caged HwTxIV-Nvoc and after illumination and (b) average normalized time courses of hNa_{V}1.6 current inhibition (n=11, mean±SEM). Scale: 2 ms, 1 nA. (c) Average normalized current at steady-state in control (dark), caged (grey) and uncaged (dark grey) conditions (n=11, ***p<0.001, repeated measures 1-way ANOVA followed by Bonferroni's post-test.). (d) Representative recording of hNa_{V}1.2 current in control (dark), caged HwTxIV-Nvoc (grey) and after illumination (dark grey). Scale: 2 ms, 1 nA. (e) Averaged normalized current at steady-state in control (dark), caged (grey) and uncaged (dark grey) conditions (n=12, mean±SEM *** p<0.001, repeated measures 1-way ANOVA followed by Bonferroni's post-test). (f-g) Controllable uncaging of HwTxIV-Nvoc analog via UV irradiation. (f) Left: Superimposition of normalized hNa_{V}1.6 currents at steady-state recorded after varying times of illumination. Middle: Average normalized time courses of hNa_{V}1.6 current inhibition (n=4-15, mean±SEM). Arrow indicates steady-state. Right: Plot of current amplitude at the end of 800 sec recording versus duration of illumination at 365 nm. Scale: 2 ms, 20% of amplitude. (g) Left: Superimposition of normalized hNa_{V}1.6 currents at steady-state recorded after varying power of illumination (4 min). Middle: Average normalized time courses of hNa_{V}1.6 current inhibition (n=7-14, mean±SEM). Arrow indicates steady-state. Right: Plot of current amplitude at the end of 800 sec recordings versus duration of illumination at 365 nm. Scale: 2 ms, 20% of amplitude.

FIG. 5: Representative examples of photocontrol (365 nm, 45 mW/cm²) of 1 nM AaHII-R⁶²K-Nvoc activity on hNa_{V}1.2 current (left), 100 nM BeKm1-Nvoc activity on hERG current (middle) and 100 nM charybdotoxin-Nvoc activity on K_{V}1.2 current (right). AaHII-R⁶²K toxin induces slowing of inactivation of hNa_{V}1.2, while BeKm1 and Charybdotoxin induce block of hERG and K_{V}1.2 channels, respectively. For AaHII-R⁶²K-Nvoc and BeKm1-Nvoc, scale is 2 ms and 1 nA. For charybdotoxin-Nvoc, scale is 400 ms and 200 pA.

FIG. 6. HwTxIV-Nvoc assessments in L5 pyramidal neurons from mouse brain slices. (a) Gray trace: AP in control solution. Black trace: the AP is inhibited by application of 500 nM non-caged HwTxIV analog (consistently observed in n=6 cells tested). (b) left: AP in control solution; centre: in the presence of 2.5 μM of caged HwTxIV-Nvoc; right: recording 1 minute after uncaging the toxin showing full AP block; the peak of the depolarization and the width of the AP used in the statistical analysis are illustrated; black traces are the control recording reported for comparison. (c) left: mean±SEM (n=8 cells) of the depolarization peak, normalized to the control values, after addition of caged HwTxIV-Nvoc (1.00±0.01) and 1 minute after photolysis (0.43±0.03). “*” indicates a significant decrease of the peak (p<0.01, paired t-test). Right: mean±SEM (n=5 cells) of the AP width, normalized to the control values, after addition of HwTxIV-Nvoc (1.18±0.11). (d) APs 1 min after uncaging of HwTxIV-Nvoc analog over a spot centered ˜100 μm from the cell body (left), and after uncaging over the soma (right). Grey traces are the control recording reported for comparison. (e) Mean±SEM (n=4 cells) of the depolarization peak, normalized to the control values, after uncaging 100 μm away from the soma (0.99±0.01) and after uncaging on the soma (0.41±0.02). “*” indicates a significant decrease of the peak (p<0.01, paired t-test). (f) Image of the UV spot (˜40 μm diameter); the dark grey curve indicates the light intensity profile; the schematic of the cell is depicted to indicate the different positions with respect to the spot and to the light intensity at which the cell was directly exposed. (g) Top: images of L5 pyramidal neuron filled with a fluorescent indicator relative to the position of a UV (405 nm) illumination spot with distance from the center indicated in each frame. Bottom: AP in the presence of caged HwTxIV-Nvoc (orange traces) and 1 minute after uncaging (dark grey traces) with the cell positioned at distances from the spot center of 100, 80, 60, 40, 20 and 0 μm. (h) Left: image of the UV spot (˜40 μm diameter); the dark grey curve indicates the light intensity profile; cells are depicted to indicate the different positions with respect to the spot. Right: mean±SEM (n=5 cells) of the depolarization peak, normalized to the control values, 1 minute after uncaging with the cell positioned at distances from the spot centre of 100 μm (1.00±0.01), 80 μm (0.99±0.01), 60 μm (0.95±0.03), 40 μm (0.77±0.12), 20 μm (0.61±0.14) and 0 μm (0.32±0.05). “*” indicates a significant decrease (p<0.01, paired t-test). (i) left, L5 pyramidal neuron filled with 500 μM of the Na⁺ indicator ING-2; the patch pipette filling the cell is visible; outlined in yellow the AIS area of Δ[Na⁺] measurement; right, images taken before and during the uncaging pulse of UV light illustrating the area of photolysis. (j) left, somatic AP (top) and associated Δ[Na⁺] signal over the red cylinder depicted in panel A (bottom) in the presence of caged HwTxIV-Nvoc; right, after uncaging the toxin, Vₘ transient depolarizing the cell to the AP peak (top) and associated Δ[Na⁺] signal (bottom). (k) mean±SEM (n=4 cells) of the Δ[Na⁺] signal maximum (peak) before and after uncaging the toxin. “*” indicates a significant decrease (p<0.01, paired t-test).

FIG. 7. In vivo photoactivation of caged HwTxIV-Nvoc. (a) Representative trajectory plots of mice injected with vehicle (left), HwTxIV (center) or caged HwTxIV-Nvoc (right). (b-c) Quantification of total movements (b) and rearing (c) in mice injected with vehicle (white circles), HwTxIV (black circles) or caged HwTxIV-Nvoc (grey circles). (n=5 in each group, mean±SEM *p<0.05*; **p<0.01; ***p<0.001 versus vehicle, repeated measures 2-way ANOVA test followed by Tukey's post-test). (d) Representative twitches obtained from mice injected with vehicle or caged HwTxIV-Nvoc at t=0; t=5; t=10; t=15; t=20 min after injection. (e) EDL twitch force normalized to muscle mass (g/mg of EDL) in vehicle or caged HwTxIV-Nvoc mice before and after illumination (365 nm, 50 mW/cm²). (n=5 in each group, mean±SEM *p<0.05*; ***p<0.001 versus t0, repeated measures Friedman test followed by Dunn's post-test).

FIG. 8. In vivo properties of non-caged and caged ω̅-conotoxin MVIIC peptides. Effects of intraperitoneal injection of 10 μg of non-caged ω̅-conotoxin MVIIC (black) and 100 μg of caged ω̅-conotoxin MVIIC (gray) versus vehicle (white) on total movements (a), distance travelled (b), inactivity time (c) and rearing (d) (n=5 in each group, mean±SEM * p<0.05; **p<0.01; 1-way ANOVA test followed by Tukey's post-test).

FIG. 9. Caging of JzTxV drastically reduces affinity of JzTxV for hNa_{V}1.4 channels. Average dose-response curves for hNaV1.4 current inhibition by non-caged JzTxV (black) and caged JzTxV (gray). The data were fitted according to a Hill equation (non-caged JzTxVIC50=50.1 nM (n=5-18) and caged JzTxV IC50=10.9 μM (n=7) (218-fold reduction in IC50)).

### DETAILED DESCRIPTION

The inventors report the development and application of a new, robust, generalizable and in vivo compatible strategy for producing photoactivatable toxins modulating ion channel and cell excitability.

### Photoactivatable Ion Channel Modulator

The present disclosure relates to a venom peptide, also herein referred as disulphide-rich venom peptide, which comprises a photolabile protection group wherein said venom peptide is a photoactivatable ion channel modulator.

By “photoactivatable ion channel modulator”, it is intended an ion channel modulator which can be activated spatially and temporally by a light emission, in particular a venom peptide comprising a photolabile protecting group wherein the photolabile group is cleaved from the venom peptide when irradiated with light.

Said photoactivatable ion channel modulator is a venom peptide which comprises a photoactivatable agent such as photolabile protecting group, also named caged group, which encapsulate said peptide in an inactive form. The caged venom peptide is thus no longer able to modulate ion channel activity at regular concentrations in comparison to non-caged venom peptide in similar condition. Illumination induces the release of the photolabile protecting group and liberates the caged peptide, permitting the venom peptide to recover its function and modulate ion channel activity.

Venom peptide also known as toxin refers to all peptides and/or proteins of any amino acid length, preferably comprising between 10 and 150 residues in either monomeric or multimeric forms derived from peptide present in animal venoms. Venom peptide include all peptides derived from animal venoms, including but not limited to isolation from crude venoms, isolation from venom gland tissues or extracts, identification based on venom gland proteome/proteomics, venome/venomics, transcriptome, and/or EST analysis. Said peptide venom can be derived as non-limiting examples from venom of a snake, cone snail, scorpion, sea anemone, lizard or spider.

Venom peptides may be directly obtained from animal venom, may be obtained by recombinant techniques or may be synthesized using standard synthetic method known to those skilled in the art.

According to the present disclosure, said venom peptide tertiary structure is complex and contains at least 1 disulphide bonds, preferably between 1 and 7 disulphide bonds.

Venom peptides according to the present disclosure are peptides which potently and selectively target ion channel.

Ion channels are pore-forming membrane proteins that allows ions to pass through the channel pore. Their functions include establishing a resting membrane potential, shaping action potentials and other electrical signals by gating the flow of ions across the cell membrane, controlling the flow of ions across secretory and epithelial cells, and regulating cell volume. Ion channel can be voltage-gated ion channel such as voltage-gated calcium ion channel, voltage-gated potassium ion channel, voltage-gated sodium ion channel, chloride channel; ligand-gated ion channel also known as ionotropic receptors such as nicotinic Acetylcholine receptor (nAChR), NMDA (N-methy-D-aspartate) receptor, or lipid-gated ion channel.

In particular, said venom peptide is an ion channel modulator. As used herein, “a modulator” refers to an activator or inhibitor. According to the present disclosure, ion channel modulator refers to a peptide which targets the ion channel and modulate its activity, in particular ion channel inhibitor or blocker impairs the conduction of ions through channels and ion channel activator facilitates ion transmission through the channel.

The term “ion channel activity” as used herein means ion current activity which can be measured for example by recording ion current flow through the channel, membrane potential changes induced by ion flow and/or accumulation or decreases in ion levels, for example sodium levels, or molecules that can flow the channel such as cobalt and fluorescent dyes. Methods to measure ion channel activity are well-known in the art and can be for example voltage-clamp and patch-clamp techniques, e.g., the “cell-attached” mode, the “inside-out” mode, and the “whole cell” mode (see, e.g., Ackerman et al., New Engl. J. Med. 336:1575-1595, 1997).

The term “modulator” or “ion channel modulator” as used herein means a compound (e.g. venom peptide) that inhibits or activates the ion channel by at least or about 30%, at least or about 40%, at least or about 50%, at least or about 60%, at least or about 70%, at least or about 80%, or at least or about 9000 compared the activity of the ion channel under similar conditions in the absence of said compound (e.g. venom peptide).

Representative examples of venom peptides with their sequences (pro- and mature peptide) and target ion channel are cited in the Table 1 below:

| | | | SEQ |
|---|---|---|---|
| Venom peptide | | | ID |
| name | Target ion channel | Sequence | NO: |
| Huwentoxin- | Voltage-gated sodium | MVNMKASMFLALAGLVLLFVVCYASESEE | 1 |
| IV-propeptide | channel inhibitor | KEFSNELLSSVLAVDDNSKGEERECLEIF | |
| | | KACNPSNDQCCKSSKLVCSRKTRWCKYQI | |
| | | GK | |
| Huwentoxin-IV | Voltage-gated sodium | ECLEIFKACNPSNDQCCKSSKLVCSRKTR | 2 |
| mature peptide | channel inhibitor | WCKYQIGK | |
| Charybdotoxin | potassium-channel | MKILSVLLLALIICSIVGWSAQFTNVSCT | 9 |
| propeptide | (hERG) inhibitor | TSKECWSVCQRLHNTSRGKCMNKKCRCYS | |
| Charybdotoxin | potassium-channel | QFTNVSCTTSKECWSVCQRLHNTSRGKCM | 10 |
| mature peptide | (hERG) inhibitor | NKKCRCYS | |
| Alpha-mammal | Voltage-gated sodium | MNYLVMISLALLFVTGVESVKDGYIVDDV | 11 |
| toxin AaHII | activator | NCTYFCGRNAYCNEECTKLKGESGYCQWA | |
| propeptide | | SPYGNACYCYKLPDHVRTKGPGRCHGR | |
| Alpha-mammal | Voltage-gated sodium | VKDGYIVDDVNCTYFCGRNAYCNEECTKL | 12 |
| toxin AaH2 | activator | KGESGYCQWASPYGNACYCYKLPDHVRTK | |
| mature peptide | | GPG RCHGR | |
| JZ-x34 (Mu- | Voltage-gated sodium | ACREWLGGCSKDADCCAHLECRKKWPYHC | 13 |
| theraphotoxin- | activator (Naᵥ 1.1)/ | VWDWTV | |
| Cg1a) | Voltage-gated sodium | | |
| | inhibitor (Naᵥ 1.4) | | |
| Delta-actitoxin- | Voltage-gated sodium | MMNRLLVFLMLGAAFMLVVSAIDQDANED | 14 |
| Avd1c (ATXII) | inhibitor (Naᵥ 1.1) | INKRGVPCLCDSDGPSVRGNTLSGIIWLA | |
| | | GCPSGWHNCKKHGPTIGWCCKQ | |
| Delta-actitoxin- | Voltage-gated sodium | GVPCLCDSDGPSVRGNTLSGIIWLAGCPS | 15 |
| Avd1c (ATXII) | inhibitor (Naᵥ 1.1) | GWHNCKKHGPTIGWCCKQ | |
| (mature peptide) | | | |
| Mu-conotoxin | Voltage-gated sodium | MKLTCVVIVAALLLTACQLITALDCGGTQ | 16 |
| GVIIJ | inhibitor (Naᵥ 1.1, | KHRALRSTIKLSLLRQHRGWCGDPGATCG | |
| | Naᵥ 1.2, Naᵥ 1.3, Naᵥ 1.4) | KLRLYCCSGFCDCYTKTCKDKSSA | |
| Mu-conotoxin | Voltage-gated sodium | WCGDPGATCGKLRLYCCSGFCDCYTKTCK | 17 |
| GVIIJ (mature | inhibitor (Naᵥ 1.1, | DKSSA | |
| peptide) | Naᵥ 1.2, Naᵥ 1.3, Naᵥ 1.4) | | |
| alpha-mammal | Voltage-gated sodium | IKDGYIVDDVNCTYFCGRNAYCNEECTKL | 18 |
| toxin Lqh-2 | inhibitor (Naᵥ 1.2) | KGESGYCQWASPYGNACYCYKLPDHVRTK | |
| | | GPGRCR | |
| PaurTx3 | Voltage-gated sodium | DCLGFLWKCNPSNDKCCRPNLVCSRKDKW | 19 |
| | inhibitor (Naᵥ 1.2) | CKYQI | |
| Mu- | Voltage-gated sodium | MKASMFLALTGLALLFVVCYASESEEKEF | 20 |
| theraphotoxin- | inhibitor (Naᵥ 1.2, | SNELLSSVLAVDDNSKGEERECLGFGKGC | |
| Hhn1b 3 | Naᵥ 1.7) | NPSNDQCCKSSNLVCSRKHRWCKYEIGK | |
| (HnTxIV) | | | |
| Mu- | Voltage-gated sodium | ECLGFGKGCNPSNDQCCKSSNLVCSRKHR | 21 |
| theraphotoxin- | inhibitor (Naᵥ 1.2, | WCKYEI | |
| Hhn1b 3 | Naᵥ 1.7) | | |
| (HnTxIV)(mature | | | |
| peptide) | | | |
| Delta-actitoxin- | Voltage-gated sodium | GGVPCLCDSDGPSVRGNTLSGIIWLAGCP | 22 |
| Afv1b (AFTII) | inhibitor (Naᵥ 1.4) | SGWHNCKAHGPTIGWCCKQ | |
| MuO-conotoxin | Voltage-gated sodium | RDCQEKWEYCIVPILGFVYCCPGLICGPF | 23 |
| MfVIA | inhibitor (Naᵥ 1.4) | VCV | |
| Delta-actitoxin- | Voltage-gated sodium | GVACRCDSDGPTVRGDSLSGTLWLTGGCP | 24 |
| Bcg1b (CGTx- | inhibitor (Naᵥ 1.5, | SGWHNCRGSGPFIGYCCKK | |
| II) | Naᵥ 1.6) | | |
| Beta/omega- | Voltage-gated sodium | YCQKWMWTCDSERKCCEGMVCRLWCKKKL | 25 |
| theraphotoxin- | inhibitor (Naᵥ 1.7) | W | |
| Tp2a (ProTx-II) | | | |
| Mu- | Voltage-gated sodium | DCLKFGWKCNPRNDKCCSGLKCGSNHNWC | 26 |
| theraphotoxin- | inhibitor (Naᵥ 1.7) | KLHI | |
| Tp1a (proTxIII) | | | |
| Beta/omega- | Voltage-gated sodium | ECRYWLGGCSAGQTCCKHLVCSRRHGWCV | 27 |
| theraphotoxin- | inhibitor (Naᵥ 1.8) | WDGTES | |
| Tp1a (ProTxI) | | | |
| Kappa- | Voltage-gated sodium | DCGTIWHYCGTDQSECCEGWKCSRQLCKY | 28 |
| sparatoxin- | inhibitor (Naᵥ 1.9) | VIDW | |
| Hv1a (HpTx1) | | | |
| omega-agatoxin | voltage-gated calcium | KKKCIAKDYGRCKWGGTPCCRGRGCICSI | 29 |
| Iva | channel inhibitor | MGTNCECKPRLIMEGLGLA | |
| | (Caᵥ2.1) | | |
| omega- | voltage-gated calcium | TRCKGKGAPCRKTMYDCCSGSCGRRGKCG | 30 |
| conotoxin | channel inhibitor | | |
| MVIIC | (Caᵥ2.1) | | |
| Huwentoxin- | voltage-gated calcium | CIGEGVPCDENDPRCCSGLVCLKPTLHGI | 31 |
| XVI | channel inhibitor | WYKSYYCYKK | |
| | (Caᵥ2.2) | | |
| omega- | voltage-gated calcium | MKLTCVVIVAVLLLTACQLITADDSRGTQ | 32 |
| conotoxin | channel inhibitor | KHRALRSTTKLSTSTRCKGKGAKCSRLMY | |
| MVIIA | (Caᵥ2.2) | DCCTGSCRSGKCG | |
| omega- | voltage-gated calcium | CKGKGAKCSRLMYDCCTGSCRSGKC | 33 |
| conotoxin | channel inhibitor | | |
| MVIIA (mature | (Caᵥ2.2) | | |
| peptide) | | | |
| omega- | voltage-gated calcium | MKLTCMVIVAVLLLTACQLITADDSRGTQ | 34 |
| conotoxin-SO3 | channel inhibitor | KHRTLRSKTKLSMSTRCKAAGKPCSRIAY | |
| | (Caᵥ 2.2) | NCCTGSCRSGKCG | |
| omega- | voltage-gated calcium | CKAAGKPCSRIAYNCCTGSCRSGKC | 35 |
| conotoxin-SO3 | channel inhibitor | | |
| (mature peptide) | (Caᵥ2.2) | | |
| Omega- | voltage-gated calcium | GVDKAGCRYMFGGCSVNDDCCPRLGCHSL | 36 |
| theraphotoxin- | channel inhibitor | FSYCAWDLTESD | |
| Hg1a (SNX- | (Caᵥ2.3) | | |
| 482) | | | |
| waglerin-1 | nicotinic acetylcholine | GGKPDLRPCHPPCHYIPRPKPR | 37 |
| | inhibitor | | |
| alpha- | nicotinic acetylcholine | ECCNPACGRHYSCGK | 38 |
| conotoxin-GI | inhibitor | | |
| alpha- | nicotinic acetylcholine | GRCCHPACGKNYSC | 39 |
| conotoxin-MI | inhibitor | | |
| alpha- | nicotinic acetylcholine | MQTAYWVMVMMMVMWITAPLSEGGKPKLI | 40 |
| conotoxin- | inhibitor | IRGLVPNDLTPQRILRSLISGRTYGIYDA | |
| PrXA | | KPPFSCAGLRGGCVLPPNLRPKEKEGR | |
| alpha- | nicotinic acetylcholine | TYGIYDAKPPESCAGLRGGCVLPPNLRPK | 41 |
| conotoxin- | inhibitor | FKE | |
| PrXA (mature | | | |
| peptide) | | | |
| Jingzhaotoxin V | Voltage-gated sodium | YCQKWMWTCDSKRACCEGLRCKLWCRKII | 42 |
| (JzTxV) | channel inhibitor | | |
| omega- | voltage-gated calcium | CKGKGAPCRKTMYDCCSGSCGRRGKC | 43 |
| conotoxin | channel inhibitor | | |
| MVIIC | (Caᵥ2.2) | | |

In a preferred embodiment said venom peptide is an inhibitor of the voltage-gated sodium ion channel. Sodium channels have important functions throughout the body. The family of sodium channel are named Naᵥ1.1 through Naᵥ1.9. For example, Naᵥ1.4 controls excitability of skeletal muscle. Naᵥ1.5 controls excitability of cardiac myocytes. Naᵥ1.1, Naᵥ1.2, and Naᵥ1.6 are abundant in the central nervous system. Naᵥ1.8 and Naᵥ1.9 are expressed in sensory neurons and have a role in pain perception. Naᵥ1.7 is broadly expressed in the peripheral nervous system and plays a role in the regulation of action potential, also in pain perception.

In a more particular embodiment, the present disclosure relates to a photoactivatable voltage-gated sodium channel inhibitor which is a venom peptide comprising a photolabile protection group, wherein said venom peptide is selected from the group consisting of: Huwentoxin-IV (SEQ ID NO: 1-8), SMT001 (jzTx-34, mu-theraphotoxin-Cg1a; UniprotKB-B1P1F7, last modified on Dec. 2, 2020, SEQ ID NO: 13), ATXII (Delta-actitoxin-Avd1c; UniProtKB-P01528, last modified on Apr. 22, 2020, SEQ ID NO: 14 or 15), Mu-conotoxin GVIIJ_{SSG} (UniProtKB-X5IWS1, last modified on Jun. 2, 2021, SEQ ID NO: 16 or 17), alpha-mammal toxin Lqh-2 (UniProtKB-X5IWS1, last modified on Dec. 2, 2020, SEQ TD NO: 18), PaurTx3 (UniProtKB-P84510(TX3_PARSR), SEQ TD NO: 19), last modified on Jun. 2, 2021), HnTxIV (Mu-theraphotoxin-Hhn1b 3, UniProtKB-D2Y2D7, last modified on Oct. 7, 2020, SEQ ID NO: 20 or 21), AFTII (Delta-actitoxin-Afv1b, UniProtKB-P10454 (NA12_ANTFU), last modified on Apr. 22, 2020, SEQ ID NO: 22), MfVIA (MuO-conotoxin MfVIA, UniProtKB-P0DM15 (CO6A_CONMF), last modified on Apr. 22, 2020, SEQ ID NO: 23), CGTx-II (Delta-actitoxin-Bcg1b, UniProtKB-P0C7P9 (NA1B_BUNCN), last modified on Feb. 26, 2020, SEQ ID NO: 24), ProTx-II (Beta/omega-theraphotoxin-Tp2a, UniProtKB-P83476 (TXPR2_THRPR), last modified on Jun. 2, 2021, SEQ ID NO: 25), ProTxIII (Mu-theraphotoxin-Tp1a, UniProtKB-P0DL64 (HPR3_THRPR), last modified on Jun. 2, 2021, SEQ ID NO: 26), ProTxI (Beta/omega-theraphotoxin-Tp1a, UniProtKB-P83480 (TXPR1_THRPR), last modified on Jun. 2, 2021, SEQ ID NO: 27), HpTx1 (Kappa-sparatoxin-Hv1a, UniProtKB-P58425, last modified on Dec. 2, 2020, SEQ ID NO: 28) and Jingzhaotoxin V (SEQ ID NO: 42), preferably selected from the amino acid sequences comprising or consisting of SEQ ID NO: 1 to 8, 13 to 28 and 42.

In a preferred embodiment, the present disclosure relates to a photoactivatable voltage-gated sodium channel inhibitor which is a disulfide-rich venom peptide comprising a photolabile protection group, wherein said venom peptide is Huwentoxin-IV.

Huwentoxin-IV (HwTx-IV), also known as mu-theraphotoxin-Hh2a is a protein of a 35-residue neurotoxin peptide (SEQ ID NO: 2) with three disulphide bridges belonging to ICK motif structural family originally isolated from the venom of the Chinese Bird Spider Haploprlma schmidti. The precursor form of Huwentoxin-IV (SEQ ID NO: 1) further comprises a Signal peptide and propeptide. The protein HwTx-IV inhibits neuronal TTX-sensitive voltage gated Na⁺ channels. It preferentially inhibits neuronal voltage-gated sodium channel subtype hNaᵥ1.7 (SCN9A, IC₅₀ is 9-26 nM), rNav1.2 (SCN2A, IC₅₀ is 150 nM), and rNaᵥ1.3 (SCN3A, IC₅₀ is 338 nM), compared with muscle subtypes rNaᵥ1.4 (SCN4A) and hNa_{V}1.5 (SCN5A) (IC₅₀ is >10 μM). Huwentoxin-IV inhibits the activation of sodium channels by trapping the voltage sensor of domain II of the site 4 in the inward, closed configuration.

In another particular embodiment, the present disclosure relates to a photoactivatable voltage-gated calcium channel inhibitor which is a disulphide-rich venom peptide comprising a photolabile protection group, wherein said venom peptide is selected from the group consisting of: omega-agatoxin Iva (UniProtKB-P30288 (TX23A_AGEAP), last modified on Jun. 2, 2021, SEQ ID NO: 29), omega-conotoxin MVIIC (UniProtKB-P37300 (O17C_CONMA), last modified on Jun. 2, 2020, SEQ ID NO: 30, or SEQ ID NO: 43), Huwentoxin-XVI (Pubchem CID: 90489025, last modified on Oct. 2, 2021, SEQ ID NO: 31), omega-conotoxin MVIIA (UniProtKB-P05484 (O17A_CONMA), last modified on Jun. 2, 2021, SEQ ID NO: 32 or 33), omega-conotoxin-SO3 (UniProtKB-Q9XZK2 (01603_CONST), last modified on Jun. 2, 2021, SEQ ID NO: 34 or 35) and SNX-482 (Omega-theraphotoxin-Hg1a, UniProtKB-P56854 (TX482_HYSGI), last modified on Dec. 11, 2019, SEQ ID NO: 36), preferably selected from the amino acid sequences comprising or consisting of SEQ ID NO: 29 to 36, more preferably omega-conotoxin MVIIC comprising or consisting of SEQ ID NO: 30 or 43.

In another particular embodiment, the present disclosure relates to a photoactivatable nicotinic acetylcholine receptor inhibitor which is a disulfide-rich venom peptide comprising a photolabile protection group, wherein said venom peptide is selected from the group consisting of: waglerin-1 (UniProtKB-P24335 (WAG13_TROWA), last modified on Jun. 2, 2021, SEQ ID NO: 37); alpha-conotoxin-GI (UniProtKB-P01519 (CA1A_CONGE), last modified on Jun. 2, 2021, SEQ ID NO: 38); alpha-conotoxin-MI (UniProtKB-P01521 (CA1_CONMA), last modified on Jun. 2, 2021, SEQ ID NO: 39) and alpha-conotoxin-PrXA (UniProtKB-P0C8S5 (CCAA_CONPI), last modified on Apr. 22, 2020, SEQ ID NO: 40 or 41), preferably selected from the amino acid sequences comprising or consisting of SEQ ID NO: 37-41.

In another particular embodiment said venom peptide is an inhibitor of the potassium ion channel. In a preferred embodiment, said potassium ion channel inhibitor is a Charybdotoxin, also named Potassium channel toxin alpha-Ktx 1.1 (UniProtKB: P13487 (KAX11_LEIHE), last modified Jun. 2, 2021 derived from deathstalker scorpion (Leiurus quinquestriatus hebraeus), preferably said venom peptide comprising or consisting of SEQ ID NO: 9 or SEQ ID NO: 10. This toxin inhibits numerous potassium channels: shaker (Ki=227 nM), Kv1.2/KCNA2 (nanomolar range), Kv1.3/KCNA3 (nanomolar range), Kv1.5/KCNA5 (Kd>100 nM), Kv1.6/KCNA6 (Ki=22 nM), KCa1.1/KCNMA1 (IC50=5.9 nM). It blocks channel activity by a simple bimolecular inhibition process. It also shows a weak interaction with nicotinic acetylcholine receptors (nAChR), suggesting it may weakly inhibit it. The mature form of charybdotoxin consists of SEQ ID NO: 10.

In another particular embodiment said venom peptide is an activator of the voltage-gated sodium ion channel. In a preferred embodiment, said sodium ion channel activator is alpha-mammal toxin AaHII (or AaH2) isolated from a Sahara scorpion Androctonus australis (UniProt-KB-P01484 (SCX_ANDAU), last modified Jun. 2, 2021), preferably said venom peptide comprising or consisting of SEQ ID NO: 11 or 12. Alpha toxins bind voltage-independently at site-3 of sodium channels (Naᵥ) and inhibit the inactivation of the activated channels, thereby blocking neuronal transmission. The toxin principally slows the inactivation process of TTX-sensitive sodium channels. The mature form of AaHII consists of SEQ ID NO: 12. It is active on rat brain Naᵥ1.2/SCN2A sodium channel (EC₅₀=2.6 nM) and on rat skeletal muscle Naᵥ1.4/SCN4A sodium channel (EC₅₀=2.2 nM), as well as on human neuronal Naᵥ1.7/SCN9A (EC₅₀=6.8 nM).

According to the present disclosure, said venom peptide can be a functional variant of the venom peptide naturally identified in the animal venom.

The term “functional variant” refers to a venom peptide variant which retains the function of the native venom peptide such as the modulation (inhibition or activation) of the target ion channel. In particular, said functional variant can bind specifically to target ion channel with a similar affinity binding, preferably said functional variant has a binding affinity measured by the EC₅₀ value similar to the native peptide, preferably varying only by a factor of 10 to 100. EC₅₀ represents the concentration of a functional variant that is required for 50% inhibition or activation of the target ion channel. The EC₅₀ can be determined by techniques known in the art, for example, by constructing a dose-response curve and examining the effect of different concentrations of the venom peptide on reversing target ion channel activity.

As used herein, the term “variant” refers to a polypeptide having an amino acid sequence having at least 70%, 75%, 80%, 85%, 90%, 95% or 99% identity to the native sequence. As used herein, the term “sequence identity” or “identity” refers to the number (%) of matches (identical amino acid residues) in positions from an alignment of two polypeptide sequences. The sequence identity is determined by comparing the sequences when aligned so as to maximize overlap and identity while minimizing sequence gaps. In particular, sequence identity may be determined using any of a number of mathematical global or local alignment algorithms, depending on the length of the two sequences. Sequences of similar lengths are preferably aligned using a global alignment algorithm (e.g. Needleman and Wunsch algorithm; Needleman and Wunsch, 1970) which aligns the sequences optimally over the entire length, while sequences of substantially different lengths are preferably aligned using a local alignment algorithm (e.g. Smith and Waterman algorithm (Smith and Waterman, 1981) or Altschul algorithm (Altschul et al, 1997; Altschul et al., 2005). Alignment for purposes of determining percent amino acid sequence identity can be achieved in various ways that are within the skill in the art, for instance, using publicly available computer software available on internet web sites such as http://blast.ncbi.nlm.nih.gov/or http://www.ebi.ac.uk/Tools/emboss/. Those skilled in the art can determine appropriate parameters for measuring alignment, including any algorithms needed to achieve maximal alignment over the full length of the sequences being compared. For purposes herein, % amino acid sequence identity values refers to values generated using the pair wise sequence alignment program EMBOSS Needle that creates an optimal global alignment of two sequences using the Needleman-Wunsch algorithm, wherein all search parameters are set to default values, i.e. Scoring matrix=BLOSUM62, Gap open=10, Gap extend=0.5, End gap penalty=false, End gap open=10 and End gap extend=0.5.

Preferably, the term “variant” refers to a polypeptide having an amino acid sequence that differs from a native sequence by less than 10, 9, 8, 7, 6, 5, 4, 3, 2 substitutions, insertions and/or deletions. In a preferred embodiment, the variant differs from the native sequence by one or more conservative substitutions, preferably by less than 10, 9, 8, 7, 6, 5, 4, 3, 2 conservative substitutions. Examples of conservative substitutions are within the groups of basic amino acids (arginine, lysine and histidine), acidic amino acids (glutamic acid and aspartic acid), polar amino acids (glutamine and asparagine), hydrophobic amino acids (methionine, leucine, isoleucine and valine), aromatic amino acids (phenylalanine, tryptophan and tyrosine), and small amino acids (glycine, alanine, serine and threonine).

Venom peptide functional variant function, in particular modulation of ion channel activity can be characterized for example by measuring voltage, current, membrane potential, and ion flux on cells or artificial membranes after treatment with said functional variant in comparison to native venom peptide. In particular embodiment, said functional variant presents a similar EC₅₀ value to the native venom peptide, preferably varying only by a factor 10 to 100 in comparison to native venom peptide under similar condition. The EC₅₀ can be determined by techniques known in the art, for example, by constructing a dose-response curve and examining the effect of different concentrations of the venom peptide on reversing target ion channel activity.

In another particular embodiment, functional variant can be tested to evaluate other types of biological effects, such as effects downstream of receptor activity. Various exemplary effects of venom peptides that may be determined using intact cells or animals include transmitter release (e.g., dopamine), hormone release (e.g., insulin), transcriptional changes, cell volume changes (e.g., in red blood cells), changes in cell metabolism such as cell growth or pH changes, and changes in intracellular second messengers such as Ca²⁺.

Functional variants may include natural variants resulting from gene polymorphism as well as artificial variant.

In a particular embodiment, said functional variant of venom peptide comprises or consists of an amino acid sequence having at least 70%, 75%, 80%, 85%, 90%, 95% or 99% identity to SEQ ID NO: 1-43.

In a more particular embodiment, said functional variant comprises or consists of an amino acid sequence having at least 70%, 75%, 80%, 85%, 90%, 95% or 99% identity to SEQ ID NO: 1-43 that differs from a native sequence by less than 5, 4, 3, 2 substitutions, insertions and/or deletions.

In a particular embodiment, said Huwentoxin-IV functional variant comprises or consists of an amino acid sequence having at least 70%, 75%, 80%, 85%, 90%, 95% or 99% identity to SEQ ID NO: 1 or 2, preferably SEQ ID NO: 2.

In a particular embodiment, said Huwentoxin-IV functional variant comprises or consists of an amino acid sequence having at least 70%, 75%, 80%, 85%, 90%, 95% or 99% identity to SEQ ID NO: 1 or 2 and comprises one or more substitution(s) selected from the group consisting of:
a substitution of glutamic acid at position 1 of SEQ ID NO: 2 to any other amino acid, in particular positive charged or uncharged amino acid, preferably positive charged amino acid such as glycine, lysine, or arginine, preferably glycine;
a substitution of glutamic acid at position 4 of SEQ ID NO: 2 to any other amino acid, in particular positive charged or uncharged amino acid, preferably positive charged amino acid such as glycine, lysine, or arginine;
a substitution of arginine at position 26 of SEQ ID NO: 2 to any other amino acid, in particular negative charged or uncharged amino acid, preferably glutamine;
a substitution of lysine at position 18 of SEQ ID NO: 2 to any other amino acid, in particular negative charged or uncharged amino acid, preferably alanine; and a combination thereof.

In particular embodiment, said huwentoxin-IV functional variant comprises or consists of an amino acid sequence having at least 70%, 75%, 80%, 85%, 90%, 95% or 99% identity to the native sequence of SEQ ID NO: 2 and comprises the substitution(s) selected from the group consisting of: E4G, E4K, E4R, E1G/E4G, E4K/R26Q and E1G/E4G/K18A.

In another term, said Huwentoxin-IV functional variant comprises or consists of an amino acid sequence having at least 70%, 75%, 80%, 85%, 90%, 95% or 99% identity to the sequence selected from the group consisting of: SEQ ID NO: 1-8.

According to the present disclosure the photoactivatable ion channel modulator is a disulphide-rich venom peptide as described above comprising a photolabile protecting group.

Photoactivatable ion channel modulator is typically generated through the grafting of a photolabile protecting group at a functional residue of the venom peptide. Thus, renders the molecule inactive, until the photolabile protecting group is removed through light-irradiation.

A photolabile protecting group (PPG), also known as photoremovable, photosensitive, photoactivatable or photocleavable protecting group or caging group refers to a chemical moiety when bound to a compound such as venom peptide according to the present disclosure, inhibits the binding and/or activity of the compound to the target ion channel and in presence of the appropriate wavelength of light is released from the compound permitting the binding and/or activity of the venom peptide.

The photolabile protecting group consists of a chromophore that can be excited through light irradiation, and subsequently induces a cleavage process. Such chromophores are typically comprised in aromatic systems such as phenyl, benzyl, quinoline, benzophenone and coumarin. Photolabile protecting groups are well-known by one skilled in the art (see for example WO2012/024558).

Typically, the photolabile protecting group (PPG) is a chromophore, in particular belonging to the following families: nitrobenzyl-based PPG, carbonyl-based PPG or benzyl-based PPG, preferably nitrobenzyl-based PPG. Nitrobenzyl-based PPG can be chosen to allow complete activation with UV light at above 340 nm. In a preferred embodiment, said PPG is compatible with the reactive function selected from the group consisting of: alcohols, thiols, amines, carboxylic acid and phosphate. In a particular embodiment, the PPG can be ortho-nitrobenzyl such as 2-(o-nitrophenyl)propyl) which is activated at 365 nm or 1-(3-nitrodibenzofuran-1-yl)ethyl which is activated at 440 nm.

However, to avoid the requirement of UV light, which can be toxic to tissues, other PPG such as coumarin-based PPG which are activated at 405 nm or near infrared (two-photon-760 nm) (e.g. coumarin lysine as described in the review Courtney T and Deiters A, Curr Opin Chem Biol. 2018 October; 46:99-107), BODIPY (Boron dipyrromethene)-based PPG or cyanine-based PPG which are activated at visible light can be used (as described in review Laia Josa-Culleré and Amadeu Llebaria, ChemPhotoChem, 5(4):296-314, 2021).

In a particular embodiment, said PPG is a coumarin-based PPG such as 7-diethylaminocoumarin-4-yl)methyl) which is activated at 405 or 470 nm, 6-bromo-7-hydroxy-4-(hydroxymethyl)coumarin which is activated at 375 nm or 7-bis(carboxymethyl)-4-(hydroxymethyl)coumarin which is activated at 380 nm.

The photolabile protecting group can be linked to the venom peptide at a suitable location on the venom peptide, in particular a functional amino acid of the venom peptide.

The term “functional amino acid or residue” or “key amino acid or residue” refers to one or several amino acids identified as being involved in the activity of the venom peptide, in particular the modulation (e.g. activation or inhibition) of ion channel as described above.

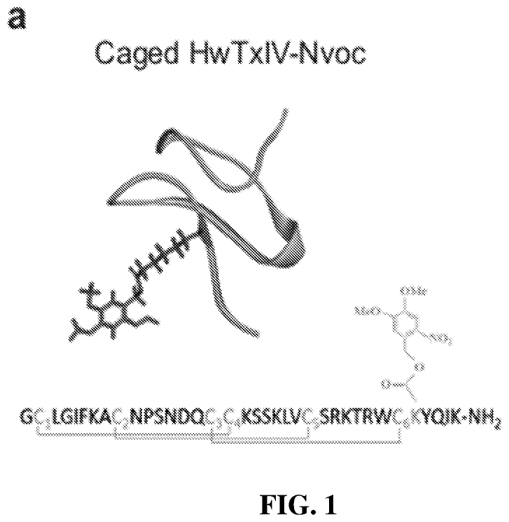

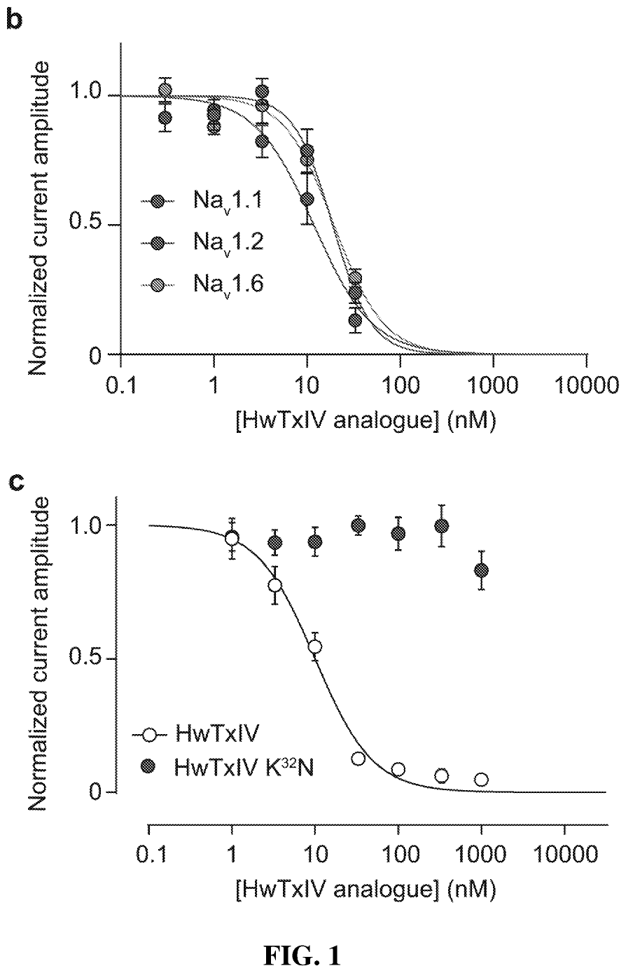

The functional amino acids of the venom peptide can be known or can be determined by one skilled in the art, for example by mutating specific amino acid of the peptide, preferably by substitution with an alanine (alanine scanning) and evaluating whether the activity of the mutated venom peptide on target ion channel is reduced in comparison to wild-type venom peptide. Ion channel activity can be measured by any methods known in the art, as described above. Preferably the functional amino acid mutation results to at least 100-fold, preferably 200, 300, 400, 500, 600, 700, 800, 1000 or over-fold increase of dose-response value of normalized ion channel current in comparison to wild-type venom peptide as measured in examples (FIG. 1c).

According to a particular embodiment, said photolabile protecting group such as nitrobenzyl-based PPG can be grafted on a functional amino acid such as tyrosine, lysine, glycine, serine or cysteine residue (Lemke, E. A. et al. Nat. Chem. Biol. 3, 769-772, 2007; Wu, N. et al. J. Am. Chem. Soc. 126, 14306-14307, 2004; Kang, J. Y et al. Neuron. 80, 358-370, 2013; Nguyen D. P. et al. J. Am. Chem. Soc. 135, 2240-2243, 2014; Uprety, R. et al. ChemBioChem, 2014; Chen PR. et al. Angew. Chem. Int. Ed. 48, 4052-4055, 2009, and for review Baker et al. ACS. Chem. Biol, 9, 1398-1407, 2014).

Non-limiting examples of photolabile protecting group amino acid that can be used according to the present disclosure may be selected from the group consisting of: O-(2-nitrobenzyl)tyrosine, S-(2-nitro-benzyl)cysteine, 4,5-dimethoxy-2-nitrobenzylGlycine, 4,5-dimethoxy-2-nitrobenzylserine (Nvoc-serine) and 4,5-dimethoxy-2-nitrobenzyllysine (Nvoc-lysine).

In some embodiment, the photoactivatable ion channel modulator according to the present disclosure comprises more than one photolabile protecting group.

In a particular embodiment, photoactivatable ion channel modulator according to the present disclosure is Huwentoxin-IV comprising or consisting of amino acid sequence selected from the group consisting of SEQ ID NO: 2-8 or a functional variant thereof wherein a lysine at position 32 is bound to a PPG as described above, preferably a nitrobenzyl-based PPG protecting group (e.g. Nvoc).

In a particular embodiment, photoactivatable ion channel modulator according to the present disclosure is a Charybdotoxin comprising or consisting of amino acid sequence SEQ ID NO: 10 or a functional variant thereof wherein a lysine at position 27, an asparagine at position 30 and/or tyrosine at position 36 is bound to a PPG as described above, preferably nitrobenzyl-based PPG protecting group (e.g. Nvoc).

In another particular embodiment, photoactivatable ion channel modulator according to the present disclosure is AaH-II comprising or consisting of amino acid sequence SEQ ID NO: 12 or a functional variant thereof wherein a residue at position 62 is bound to a PPG as described above, preferably nitrobenzyl-based PPG protecting group (e.g. Nvoc). In a particular embodiment, an arginine at position 62 is replaced by a lysine which is bound to a PPG as described above.

In another particular embodiment, photoactivatable ion channel modulator according to the present disclosure is omega-conotoxin MVIIC comprising or consisting of amino acid sequence SEQ ID NO: 43 or a functional variant thereof wherein a lysine at position 2 is bound to a PPG as described above, preferably a nitrobenzyl-based PPG protecting group (e.g. Nvoc).

In another particular embodiment, photoactivatable ion channel modulator according to the present disclosure is Jingzhaotoxin V comprising or consisting of amino acid sequence SEQ ID NO: 42 or a functional variant thereof wherein a lysine at position 22 is bound to a PPG as described above, preferably a nitrobenzyl-based PPG protecting group (e.g. Nvoc).

### Method for Synthesis of Photoactivatable Peptide

Disulphide-rich venom peptide comprising photolabile protecting group as described herein can be synthesized using standard synthetic methods known to those skilled in the art, for example chemical synthesis.

In a preferred embodiment, disulphide-rich venom peptides are obtained by stepwise condensation of amino acid residues, either by condensation of a preformed fragment already containing an amino acid sequence in appropriate order, or by condensation of several fragments previously prepared, while protecting the amino acid functional groups except those involved in peptide bond during condensation. In particular, the peptides can be synthesized according to the method originally described by Merrifield.

Examples of chemical synthesis technologies are solid phase synthesis and liquid phase synthesis. As a solid phase synthesis, for example, the amino acid corresponding to the C-terminus of the peptide to be synthesized is bound to a support which is insoluble in organic solvents, and by alternate repetition of reactions, one wherein amino acids with their amino groups and side chain functional groups protected with appropriate protective groups are condensed one by one in order from the C-terminus to the N-terminus, and one where the amino acids bound to the resin or the protective group of the amino groups of the peptides are released, the peptide chain is thus extended in this manner. Solid phase synthesis methods are largely classified by the tBoc method and the Fmoc method, depending on the type of protective group used. Typically used protective groups include tBoc (t-butoxycarbonyl), Cl—Z (2-chlorobenzyloxycarbonyl), Br—Z (2-bromobenzyloyycarbonyl), Bzl (benzyl), Fmoc (9-fluorenylmcthoxycarbonyl), Mbh (4, 4′-dimethoxydibenzhydryl), Mtr (4-methoxy-2, 3, 6-trimethylbenzenesulphonyl), Trt (trityl), Tos (tosyl), Z (benzyloxycarbonyl) and Clz-Bzl (2, 6-dichlorobenzyl) for the amino groups; N02 (nitro) and Pmc (2,2, 5,7, 8-pentamethylchromane-6-sulphonyl) for the guanidino groups); and tBu (t-butyl) for the hydroxyl groups). After synthesis of the desired peptide, it is subjected to the de-protection reaction and cut out from the solid support. Such peptide cutting reaction may be carried with hydrogen fluoride or tri-fluoromethane sulfonic acid for the Boc method, and with TFA for the Fmoc method.

One another method for incorporation of a photocaged amino acid into a nascent protein involves misaminoacylation of tRNA. Normally, a species of tRNA is charged by a single, cognate native amino acid. This selective charging, termed here enzymatic aminoacylation, is accomplished by enzymes called aminoacyl-tRNA synthetases and requires that the amino acid to be charged to a tRNA molecule be structurally similar to a native amino acid. Chemical misaminoacylation can be used to charge a tRNA with a non-native amino acid such as photocaged amino acids.

The N- and C-termini of the peptides described herein may be optionally protected against proteolysis. For instance, the N-terminus may be in the form of an acetyl group, and/or the C-terminus may be in the form of an amide group. Internal modifications of the peptides to be resistant to proteolysis are also envisioned, e.g. wherein at least a —CONH— peptide bond is modified and replaced by a (CH₂NH) reduced bond, a (NHCO) retro-inverso bond, a (CH₂-0) methylene-oxy bond, a (CH₂—S) thiomethylene bond, a (CH₂CH₂) carba bond, a (CO—CH₂) cetomethylene bond, a (CHOH—CH₂) hydroxyethylene bond), a (N—N) bound, a E-alcene bond or also a —CH═CH-bond.

For instance, the peptide may be modified by acetylation, acylation, amidation, cross-linking, cyclization, disulfide bond formation, formation of covalent cross-links, formation of cysteine, formation of pyroglutamate, formylation, gamma-carboxylation, glycosylation, GPI anchor formation, hydroxylation, iodination, methylation, myristylation, oxidation, phosphorylation, and the like.

The peptides of the invention may be composed of amino acid(s) in D configuration, which render the peptides resistant to proteolysis. They may also be stabilized by intramolecular crosslinking, e.g. by modifying at least two amino acid residues with olefinic side chains, preferably C3-C8 alkenyl chains, preferably penten-2-yl chains) followed by chemical crosslinking of the chains, according to the so-called “staple” technology described in Walensky et al, 2004. For instance, amino acids at position i and i+4 to i+7 can be substituted by non-natural amino acids that show reactive olefinic residues. All these proteolysis-resistant chemically modified peptides are encompassed in the present disclosure.

In another aspect of the invention, peptides are covalently bound to a polyethylene glycol (PEG) molecule by their C-terminal terminus or a lysine residue, notably a PEG of 1500 or 4000 MW, for a decrease in urinary clearance and in therapeutic doses used and for an increase of the half-life in blood plasma. In yet another embodiment, peptide half-life is increased by including the peptide in a biodegradable and biocompatible polymer material for drug delivery system forming microspheres. Polymers and copolymers are, for instance, poly(D,L-lactide-co-glycolide) (PLGA) (as illustrated in US2007/0184015, SoonKap Hahn et al).

### Pharmaceutical Composition

In a further aspect, the present disclosure also provides a pharmaceutical composition comprising a photoactivatable ion channel modulator as described above and a pharmaceutically acceptable excipient.

The pharmaceutically acceptable excipient is selected according to the route of administration and the nature of the active ingredient, e.g. a peptide, a nucleic acid or a vector expression. As used herein, the term “pharmaceutically acceptable” means approved by a regulatory agency or recognized pharmacopeia such as European Pharmacopeia, for use in animals and/or humans. The term “excipient” refers to a diluent, adjuvant, carrier, or vehicle with which the therapeutic agent is administered. As is well known in the art, pharmaceutically acceptable excipients are relatively inert substances that facilitate administration of a pharmacologically effective substance and can be supplied as liquid solutions or suspensions, as emulsions, or as solid forms suitable for dissolution or suspension in liquid prior to use. For example, an excipient can give form or consistency, or act as a diluent. Suitable excipients include but are not limited to stabilizing agents, wetting and emulsifying agents, salts for varying osmolality, encapsulating agents, pH buffering substances, and buffers. Such excipients include any pharmaceutical agent suitable for direct delivery to the eye which may be administered without undue toxicity.

The pharmaceutical composition is formulated for administration by a number of routes, including but not limited to oral, parenteral, intraocular and local.

The pharmaceutically acceptable carriers are those conventionally used. The pharmaceutical composition comprises a therapeutically effective amount of the compound, e.g., sufficient to show benefit to the individual to whom it is administered. The pharmaceutically effective dose depends upon the composition used, the route of administration, the type of mammal (human or animal) being treated, the physical characteristics of the specific mammal under consideration, concurrent medication, and other factors, that those skilled in the medical arts will recognize.

Possible pharmaceutical compositions include those suitable for oral, rectal, topical, intraocular or parenteral administration. For these formulations, conventional excipient can be used according to techniques well known by those skilled in the art.

Preferably, the pharmaceutical composition is suitable for transcutaneous, transmucosal or intraocular injection.

Pharmaceutical compositions according to the invention may be formulated to release the active drug substantially immediately upon administration or at any predetermined time or time period after administration.

### Therapeutic Use

The photoactivatable ion channel modulator according to the present disclosure is particularly useful for the treatment of a disorder by the control release of the venom peptide at a selected site, protecting other areas of the patient's body from the biological effect of the venom peptide. Indeed, after general administration of the photoactivatable ion channel modulator to the patient, the site to be treated is exposed to appropriate radiation releasing venom peptide which can modulate target ion channel activity locally and at specific time in said patient.

The present disclosure also relates to the photoactivatable ion channel modulator according to the present disclosure for use as medicament, preferably for use in the treatment of ion channels-related disease, in particular which can be treated by targeting ion channel activity in a patient.

In a particular embodiment, said ion channels-related disease can be selected from the group consisting of: central nervous system disease such as epileptic syndromes, ataxia syndromes, familial hemiplegic migraine or Dravet syndrome; heart disease such as Long QT and short QT syndromes, Brugada syndromes or catecholaminergic polymorphic ventricular tachycardia; pancreas disease such as familial congenital hyperinsulinism and neonatal diabetes mellitus, skeletal muscle disease such as non-dystrophic myotonias or periodic paralysis; bone disease such as osteopetrosis, kidney disease such as Bartter's syndrome, Dent disease or EAST/SESAME syndrome and peripheral nervous system disease such as pain syndrome or neuropathies.

In a preferred embodiment, the present disclosure relates to the photoactivatable ion channel modulator according to the present disclosure for use for the treatment of a disease caused by ion channel dysfunction.

Diseases caused by ion channel dysfunction, are diseases caused by disturbed function of ion channel subunits or the proteins that regulate them. These diseases may be either congenital and for example results from mutation(s) in the encoding genes or acquired for example resulting from autoimmune attack on an ion channel.

Diseases associated with dysfunction of an ion channel might be caused by genetic dysfunction of voltage- or ligand-gated ion channels, such as voltage-gated calcium channels, voltage-gated sodium channels, voltage-gated potassium channels, voltage-gated chloride channels, nicotinic acetylcholine receptors, ryanodine receptors (calcium release channels), cyclic nucleotide-gated receptors, ATP-receptors, GABA-A receptors, glutamate-NMDA receptors, glycine-receptors, 5-HT3-receptors, and pH sensitive channels such as acid-sensing ion channel (ASIC), and TRP receptors.

In a particular embodiment, said ion channel-related disease according to the present disclosure is caused by abnormal high membrane cell excitability, and preferably is selected from the group consisting: epilepsy, Dravet syndrome, convulsion, cardiac arrythmia, pain syndrome, erythromelalgia, lumbosacral radiculopathy and trigeminal neuralgia.

In a particular embodiment, the present disclosure relates to a photoactivatable ion channel modulator for use in the treatment of a ion channel-related disease, preferably caused by abnormal high membrane cell excitability as described above, wherein said photoactivatable ion channel modulator is selected from the group consisting of: photoactivatable voltage-gated ion channel inhibitor such as photoactivatable voltage-gated sodium channel inhibitor, photoactivatable nicotinic acetylcholine inhibitor, photoactivable calcium channel inhibitor and photoactivable potassium channel inhibitor.

In a preferred embodiment, said photoactivatable voltage-gated sodium channel inhibitor is a disulphide-rich venom peptide comprising a photolabile protecting group, wherein said venom peptide is selected from the group consisting of: huwentoxin-IV (SEQ ID NO: 1-8), SMT001 (Jz-Tx34, mu-theraphotoxin-Cg1a, UniProtKB-B1P1F7, last modified on Dec. 2, 2020, SEQ ID NO: 13), ATXII (Delta-actitoxin-Avd1c; UniProtKB-P01528, last modified on Apr. 22, 2020, SEQ ID NO: 14 or 15), Mu-conotoxin GVIIJ_{SSG} (UniProtKB-X5IWS1, last modified on Jun. 2, 2021, SEQ ID NO: 16 or 17), alpha-mammal toxin Lqh-2 (UniProtKB-X5IWS1, last modified on Dec. 2, 2020, SEQ ID NO: 18), PaurTx3 (UniProtKB-P84510(TX3_PARSR), last modified on Jun. 2, 2021, SEQ ID NO: 19), HnTxIV (Mu-theraphotoxin-Hhn1b 3, UniProtKB-D2Y2D7, last modified on Oct. 7, 2020, SEQ ID NO: 20 or 21), AFTII (Delta-actitoxin-Afv1b, UniProtKB-P10454 (NA12_ANTFU), last modified on Apr. 22, 2020, SEQ ID NO: 22), MfVIA (MuO-conotoxin MfVIA, UniProtKB-P0DM15 (CO6A_CONMF), last modified on Apr. 22, 2020, SEQ ID NO: 23), CGTx-II (Delta-actitoxin-Bcg1b, UniProtKB-P0C7P9 (NA1B_BUNCN), last modified on Feb. 26, 2020, SEQ ID NO: 24), ProTx-II (Beta/omega-theraphotoxin-Tp2a, UniProtKB-P83476 (TXPR2_THRPR), last modified on Jun. 2, 2021, SEQ ID NO: 25), ProTxIII (Mu-theraphotoxin-Tp1a, UniProtKB-P0DL64 (HPR3_THRPR), last modified on Jun. 2, 2021, SEQ ID NO: 26), ProTxI (Beta/omega-theraphotoxin-Tp1a, UniProtKB-P83480 (TXPR1_THRPR), last modified on Jun. 2, 2021, SEQ ID NO: 27), HpTx1 (Kappa-sparatoxin-Hv1a, UniProtKB-P58425, last modified on Dec. 2, 2020, SEQ ID NO: 28), and Jingzhaotoxin V (SEQ ID NO: 42) or functional variant thereof, preferably said venom peptide is selected among amino acid sequences comprising or consisting of SEQ ID NO: 1 to 8, 13 to 28 and 42 or functional variant thereof, more preferably Huwentoxin-IV (SEQ ID NO: 1-8) or functional variant thereof. Voltage-gated sodium (Na_{V}) channels are crucial in the initiation and propagation of electrical signals (action potentials) in excitable neuronal cells, muscles, and heart tissues. In a more preferred embodiment, the present disclosure relates to a photoactivatable voltage-gated sodium (Na_{V}) channel inhibitor as defined above (e.g. Huwentoxin-IV comprising a PPG) for use in the treatment of a disease caused by abnormal cell excitability, preferably selected from the group consisting of: epilepsy, Dravet syndrome, convulsion, familial hemiplegic migraine, cardiac arrythmia, pain, erythromelalgia, lumbosacral radiculopathy and trigeminal neuralgia.

In another preferred embodiment, said photoactivatable voltage-gated calcium channel inhibitor is a disulphide-rich venom peptide comprising a photolabile protecting group, wherein said venom peptide is selected from the group consisting of: omega-agatoxin Iva (UniProtKB-P30288 (TX23A_AGEAP), last modified on Jun. 2, 2021) (SEQ ID NO; 29), omega-conotoxin MVIIC (UniProtKB-P37300 (O17C_CONMA), last modified on Jun. 2, 2020 (SEQ ID NO: 30 or SEQ ID NO: 43), Huwentoxin-XVI (Pubchem CID: 90489025, last modified on Oct. 2, 2021) (SEQ ID NO: 31), omega-conotoxin MVIIA (UniProtKB-P05484 (017A_CONMA), last modified on Jun. 2, 2021) (SEQ ID NO: 32 or 33), omega-conotoxin-S03 (UniProtKB-Q9XZK2 (01603_CONST), last modified on Jun. 2, 2021) (SEQ ID NO: 34 or 35), SNX-482 (Omega-theraphotoxin-Hg1a, UniProtKB-P56854 (TX482_HYSGI), last modified on Dec. 11, 2019) (SEQ ID NO: 36), preferably said venom peptide is selected among amino acid sequences comprising or consisting of SEQ ID NO: 29 to 36 and 43 or functional variant thereof, more preferably omega-conotoxin comprising or consisting of SEQ ID NO: 30 or 43 or functional variant thereof.

In another preferred embodiment, said photoactivatable nicotinic acetylcholine inhibitor is a disulphide-rich venom peptide comprising a photolabile protecting group, wherein said venom peptide is selected from the group consisting of: waglerin-1 (UniProtKB-P24335 (WAG13_TROWA), last modifier on Jun. 2, 2021) (SEQ ID NO: 37); alpha-conotoxin-GI (UniProtKB-P01519 (CA1A_CONGE), last modified on Jun. 2, 2021) (SEQ ID NO: 38); alpha-conotoxin-MI (UniProtKB-P01521 (CA1_CONMA), last modified on Jun. 2, 2021) (SEQ ID NO: 39); and alpha-conotoxin-PrXA (UniProtKB-P0C8S5 (CCAA_CONPI), last modified on Apr. 22, 2020) (SEQ ID NO: 40 or 41) or functional variant thereof, preferably said venom peptide is selected among amino acid sequences comprising or consisting of SEQ ID NO: 37 to 41 or functional variant thereof

In another preferred embodiment, said photoactivatable voltage-gated potassium channel inhibitor is a disulphide-rich venom peptide comprising a photolabile protecting group, wherein said venom peptide is dendrotoxin which are synaptic neurotoxins produced by mamba snakes (Dendroaspis) that block particular subtypes of voltage-gated potassium channels in neurons, thereby enhancing the release of acetylcholine at neuromuscular junctions. In another particular embodiment, said ion channel-related disease is neuromuscular junction disorder and is selected from the group consisting of: skeletal muscle disorder such as myotonia and paralyses, ataxias, myasthenia and hyperthermia,

In a particular embodiment, the present disclosure relates to a photoactivatable ion channel modulator for use in the treatment of neuromuscular junction disorder as described above, wherein said photoactivatable ion channel modulator is selected from the group consisting of photoactivatable voltage-gated sodium channel activator, photoactivatable voltage-gated potassium channel activator and photoactivatable voltage-gated calcium channel activator.

In a more particular embodiment, said neuromuscular junction disorder can be acquired neuromuscular junction disorder such as Myasthenia gravis, autoimmune neuromyotonia and Lambert-Eaton syndrome; or congenital and familial neuromuscular disorders such as congenital myasthenia gravis syndrome.

The disclosure also provides a method for treating a disease as described above in a patient in need thereof comprising administering to a patient a therapeutically effective amount of the photoactivatable ion channel modulator or pharmaceutical composition thereof as described above and applying light irradiation to said patient at the appropriate wavelength.

Preferably, the radiation applied is UV, visible or IR radiation of the wavelength between about 200 nm to about 1,000 nm, more preferably between about 260 nm to about 600 nm, and more preferably between about 300 nm to about 500 nm. Radiation is administered continuously or as pulses for hours, minutes or seconds, and preferably for the shortest amount of time possible to minimize any risk of damage to the substrate and for convenience. Visible, UV and IR radiation are also preferred as all three of these forms of radiation can be conveniently and inexpensively generated from commercially available sources. The patient may be irradiated at specific localisation (specific tissue, region of the body) and at a specific time. In a preferred embodiment, said photoactivatable ion channel modulator is a disulphide-rich venom peptide comprising a nitrobenzyl-based PPG (e.g. Nvoc) and said photoactivatable ion channel modulator is activated by irradiating said tissue at UV light above 340 nm.

By “therapeutically effective amount” refers to an amount effective, at dosages and for periods of time necessary to achieve the desired therapeutic result. The therapeutically effective amount of the product of the disclosure or pharmaceutical composition that comprises it may vary according to factors such as the disease state, age, sex, and weight of the individual, and the ability of the product or pharmaceutical composition to elicit a desired response in the individual. Dosage regimens may be adjusted to provide the optimum therapeutic response. A therapeutically effective amount is also typically one in which any toxic or detrimental effect of the product or pharmaceutical composition is outweighed by the therapeutically beneficial effects.

As used herein, the term “patient” or “individual” denotes a mammal. Preferably, a patient or individual according to the disclosure is a human.

In the context of the disclosure, the term “treating” or “treatment”, as used herein, means reversing, alleviating or inhibiting the progress of the ion channel-related disease or condition to which such term applies, or reversing, alleviating or inhibiting the progress of one or more symptoms of the disorder or condition to which such term applies.

The product of the present disclosure is generally administered according to known procedures, at dosages and for periods of time effective to induce a therapeutic effect in the patient.

The administration can be systemic or local. Systemic administration is preferably parenteral such as subcutaneous (SC), intramuscular (IM), intravascular such as intravenous (IV) or intraarterial; intraperitoneal (IP); intradermal (ID), interstitial or else. The administration may be for example by injection or perfusion. In some preferred embodiments, the administration is parenteral, preferably intravascular such as intravenous (IV) or intraarterial. The practice of the present disclosure will employ, unless otherwise indicated, conventional techniques, which are within the skill of the art. Such techniques are explained fully in the literature.

In preferred embodiment, said administration is transcutaneous, transmucosal or ocular administration.

The disclosure also provides use of photoactivatable ion channel modulator or pharmaceutical composition thereof as described above in the manufacture of a medicament for the treatment of a ion channel-related disease as defined above.

### Non-Therapeutic Use

In another embodiment, the present disclosure relates to a non-therapeutic use of a photoactivatable ion channel modulator as described above for modulating activity of ion channel in a tissue of a subject wherein said photoactivatable ion channel modulator is activated by irradiating said tissue at the appropriate wavelength. In a preferred embodiment, said photoactivatable ion channel modulator is a disulphide-rich venom peptide comprising a nitrobenzyl-based PPG (e.g. Nvoc) and said photoactivatable ion channel modulator is activated by irradiating said tissue at UV light above 340 nm.

In a preferred embodiment, the present disclosure relates to a non-therapeutic use of a photoactivatable ion channel modulator as described above, preferably photoactivatable voltage-gated sodium channel inhibitor (e.g. Huwentoxin-IV comprising PPG) for preventing cell neuromuscular signal propagation in a subject, preferably for modifying soft-tissue features in a subject, again more preferably for reducing wrinkles. In a preferred embodiment, said ion channel modulator can be administered via transcutaneous or transmucosal injection. In a particular embodiment, the photoactivatable ion channel modulator is administered to the face or neck of the subject.

Said photoactivatable ion channel modulator is thereafter activated by irradiating said tissue at the appropriate wavelength, preferably wherein said photoactivatable ion channel modulator is a disulphide-rich venom peptide comprising nitrobenzyl-based PPG (e.g. Nvoc) and said photoactivatable ion channel modulator is activated by irradiating said tissue at UV light above 340 nm.

In another particular embodiment, the present disclosure relates to said photoactivatable ion channel modulator as described above for use in a cosmetic method for preventing cell neuromuscular signal propagation in a subject, preferably for modifying soft-tissue features (e.g. reducing wrinkles) wherein the administration of said photoactivatable ion channel modulator involved a surgical step such as transcutaneous or transmucosal injection and wherein said tissue is irradiated at the appropriate wavelength. In a preferred embodiment, said photoactivatable ion channel modulator is a disulphide-rich venom peptide comprising nitrobenzyl-based PPG (e.g. Nvoc) and said photoactivatable ion channel modulator is activated by irradiating said tissue at UV light above 340 nm.

In a further aspect, the present disclosure also concerns photoactivatable ion channel modulator as described above for use for modulating activity of ion channel, for example for in vitro diagnostic reagent, drug screening reagent or research tool.

The following examples are given for purposes of illustration and not by way of limitation.

### EXAMPLES

### Methods

### Molecular Modelling

To understand if modifications in toxin activity are connected to alterations in peptide/channel interaction, molecular simulations were performed. The NMR structure of Huwentoxin-IV (HwTxIV) (pdb code 1MB6) and VSD2-NaᵥAb (pdb code 6N4R) were used as a template for building analogues and model of HwTxIV binding on VSD2-NavAb using Discovery Studio (Dassault System). The plugin, “Show bumps”, was implemented in PyMOL to calculate the van-der Waals (vdW) repulsion between HwTxIV analogues and VSD2-NavAb (http://www.pymolwiki.org/index.php/Show_bumps).

### Chemical Syntheses of HwTxIV and Analogues

All peptides were assembled stepwise using Fmoc-based Solid Phase Peptide Synthesis (SPPS) on a PTI Symphony synthesizer at a 0.05 mmol or 0.1 mmol scale on 2-chlorotrityl chloride polystyrene resin (substitution approximately 1.6 mmol/g). The Fmoc protecting group was removed using 20% piperidine in DMF and free amine was coupled using tenfold excess of Fmoc amino acids and HCTU/DIEA activation in NMP/DMF (3×15 min). Linear peptides were de-protected and cleaved from the resin with TFA/H₂O/1,3-dimethoxybenzene(DMB)/TIS/2,2′-(Ethylenedioxy)diethanethiol(DODT) 85.1/5/2.5/3.7/3.7 (vol.), then precipitated out in cold diethyl ether. The resulting white solids were washed twice with diethyl ether, re-suspended in H₂O/acetonitrile and freeze dried to afford crude linear peptide. Oxidative folding of the crude linear toxin analogue was successfully conducted at RT in the conditions optimized for the HwTxIV analog using a peptide concentration of 0.1 mg/mL in a 0.1 M Tris buffer at pH 8.0 containing 10% of DMSO.

### Monitoring of Nvoc Deprotection by Analytical RP-HPLC

Analytical RP-HPLC was performed using an SPD M20-A system (Shimadzu) with a Luna OmegaPS C18 column (4.6×250 mm, 5 μm, 100 Å). 20 μL (corresponding to 7 μg of material) was loaded and a 5-60% acetonitrile gradient (0.1% TFA v/v) was applied over 35 min at room temperature to detect analytes by UV absorbance at 214 nm. Illumination of samples was performed at 365 nm for different times (between 1 sec and 30 min illumination time) at 41.8 mW/cm² or less (as specified in the Result section) for 10 min using a CoolLED pE4000 light source (CoolLED, UK). Flash energy has been measured using a High Sensitive Thermal Power Head (S401C, ThorLabs). Coelution of uncaged HwTxIV-Nvoc and non-caged HwTxIV analogs was performed using a 50:50 ratio for both compounds.

### NMR Spectrometry

Caged HwTxIV-Nvoc analog was illuminated for 30 min at 100% power (45 mW/cm2) using a CoolLED pE4000 light source (CoolLED, UK) as described previously. Three 200 μL solutions were used in 3 mm NMR tubes for i) the caged HwTxIV-Nvoc before illumination (500 mM); ii) the caged HwTxIV-Nvoc after illumination (500 μM); and iii) the non-caged HwTxIV (200 mM). For each sample, two-dimensional homonuclear (80 ms TOCSY and 160 ms NOESY) and heteronuclear ¹³C-HSQC (natural abundancy) spectra were acquired on a BRUKER 700 MHz NMR spectrometer equipped with a 5 mm TCI cryoprobe, at 298K. Processing and analyses were performed with Bruker's TopSpin3.2 and CcpNMR programs. Spectra are drawn with CcpNMR program. 3D structure is drawn with the PyMOL software (The PyMOL Molecular Graphics System, Version 2.0 Schrödinger, LLC).

### Cell Cultures

HEK293 cells stably expressing the human (h) Na_{V}1.1, Na_{V}1.2 or Na_{V}1.6 channels, CHO cells transiently expressing K_{V}1.2 and CHO cells stably expressing the hERG channels were cultured in Dulbecco's Modified Eagle's Medium (DMEM) supplemented with 10% fetal bovine serum, 1 mM pyruvic acid, 4.5 g/L glucose, 4 mM glutamine, 800 μg/mL G418, 10 U/mL penicillin and 10 μg/mL streptomycin (Gibco, Grand Island, NY). All cell lines were incubated at 37° C. in a 5% CO₂ atmosphere. For electrophysiological recordings, cells were detached with trypsin and floating single cells were diluted (˜300,000 cells/mL) in medium contained (in mM): 140 mM, 4 KCl, 2 CaCl₂, 1 MgCl₂, 5 glucose and 10 HEPES (pH 7.4, osmolarity 298 mOsm).

### Automated Patch-Clamp Recordings and Pharmacological Studies

Whole-cell recordings were used to investigate the effects of HwTx-IV analogs on HEK293 cells expressing hNa_{V}1.1, hNa_{V}1.2, hNav1.4 or hNa_{V}1.6 channels but also the effects of AaHIIR⁶²K-Nvoc, BeKm1-Nvoc and charybdotoxin-Nvoc analogs on hNa_{V}1.2, hERG and hK_{V}1.2, respectively. Automated patch-clamp recordings were performed using the SyncroPatch 384PE from Nanion (München, Germany). Chips with single-hole medium resistance of 4.52±0.08 MΩ (n=384) were used for recordings. Pulse generation and data collection were performed with the PatchControl384 v1.5.2 software (Nanion) and the Biomek v1.0 interface (Beckman Coulter). Whole-cell recordings were conducted according to the recommended procedures of Nanion. Cells were stored in a cell hotel reservoir at 10° C. with shaking speed at 60 RPM. After initiating the experiment, cell catching, sealing, whole-cell formation, liquid application, recording, and data acquisition were all performed sequentially and automatically. For sodium currents, the intracellular solution contained (in mM): 10 CsCl, 110 CsF, 10 NaCl, 10 EGTA and 10 HEPES (pH 7.2, osmolarity 280 mOsm). For potassium currents, the intracellular solution contained (in mM): 10 KCl, 110 KF, 10 NaCl, 10 EGTA and 10 HEPES (pH 7.2, osmolarity 280 mOsm). For concentration-response and photoactivation experiments, the extracellular solution contained (in mM): 140 NaCl, 4 KCl, 2 CaCl₂, 1 MgCl₂, 5 glucose and 10 HEPES (pH 7.4, osmolarity 298 mOsm). For sodium currents, whole-cell experiments were performed at a holding potential of −100 mV at room temperature (18-22° C.). Currents were sampled at 20 kHz. Each peptide was prepared at various concentrations in the extracellular solution, itself supplemented with 0.3% bovine serum albumin (BSA). A single toxin concentration was applied to each cell. The working compound solution was diluted 3 times in the patch-clamp recording well by adding 30 to 60 μl of external solution to reach the final reported concentration and the test volume of 90 μl. For establishing dose-response curves, the compounds were tested at a test potential of 0 mV for 50 ms with a pulse every 5 sec. The percentage of current inhibition by the peptides was measured at equilibrium of blockage or at the end of a 14-min application time. For hERG current, whole-cell experiments were performed at a holding potential of −80 mV and BeKm1-Nvoc was tested at a 200 ms test potential of +60 mV following a first activation step of 1000 ms at +60 mV and a 10 ms step at −120 to recover from inactivation with a pulse every 8 sec. For K_{V}1.2 current, whole-cell experiments were performed at a holding potential of −90 mV and charybdotoxin-Nvoc was tested at a 2000 ms test potential of +60 mV with a pulse every 12 sec.

### Photoactivation of HwTxIV Analogs on HEK293 Cells

The pharmacology of the caged HwTxIV analog, as well as the efficiency of the released product to block Naᵥ channels was studied using combined automated patch-clamp and UV illumination. After 2 min of control, caged HwTxIV-Nvoc was added to the external buffer and effects were recorded for 100 s prior to a 250 s-duration illumination for photocleavage induction of the caged compound. Different wavelengths, illumination powers' and durations were used as specified in figure legends. For AaHIIR⁶²K-Nvoc, BeKm1-Nvoc and Charybdotoxin-Nvoc, a 250 s-duration illumination at 45 mW/cm² was used to uncaged compounds.

Construction of hNaᵥ1.6/Kᵥ2.1 Chimeras

Channel chimeras were generated using sequential PCR with K_{V}2.1Δ7^{46,47} (Genscript, USA) and hNa_{V}1.6 (NM_014191, Origene Technologies, USA) as templates. cRNA was synthesized using T7 polymerase (mMessage mMachine kit, Thermo Fisher, USA) after linearizing cDNA with appropriate restriction enzymes. This chimeric approach was previously shown to robustly indicate the binding locus of toxins (Bosmans, F., Martin-Eauclaire, M. F. & Swartz, K. J. Nature 456, 202-208).

### Two-Electrode Voltage-Clamp Recordings

Two-electrode voltage-clamp recording techniques on Xenopus laevis oocytes (OC-725C, Warner Instruments, USA; 150 μL recording chamber) were used to measure channel currents 1 day after cRNA injection and incubation at 17° C. in ND96 that contained (in mM): 96 NaCl, 2 KCl, 5 HEPES, 1 MgCl₂ and 1.8 CaCl₂, 50 μg/mL gentamycin, pH 7.6. Data were filtered at 4 kHz and digitized at 20 kHz using pClamp software (Molecular Devices, USA). Microelectrode resistances were 0.5-1 MΩ when filled with 3 M KCl. For K_{V} channel experiments, the external recording solution contained (in mM): 50 KCl, 50 NaCl, 5 HEPES, 1 MgCl₂ and 0.3 CaCl₂, pH 7.6 with NaOH. All experiments were performed at room temperature (˜21° C.) and toxin samples were diluted in recording solution with 0.1% BSA. Voltage-activation relationships were obtained by measuring tail currents for K_{V} channels. After addition of toxin to the recording chamber, equilibration between toxin and channel was monitored using weak depolarizations elicited at 5-10 s intervals. For all channels, voltage-activation relationships were recorded in the absence and presence of toxin. Off-line data analysis was performed using Clampfit 11 (Molecular Devices, USA) and Origin 8 (Originlab, USA).

### Mice

For coronal slices, procedures were reviewed by the ethics committee affiliated to the animal facility of the university (D3842110001) and performed in accordance with European Directives 2010/63/UE on the care, welfare, and treatment of animals. Specifically, C57BL/6J mice were housed with their mother with ad libitum access to food and water. Animals (21-35 postnatal days old) were anesthetised by isoflurane inhalation and the entire brain was removed after decapitation. For neuromuscular experiments, experiments were carried out on 25 male mice (C57BL/6J mice) aged between 6 to 8 weeks. Mice were housed 5 per cage and maintained on a 12/12 h light/dark schedule in a temperature-controlled facility (22±1° C.) with free access to food and water. Animals were kept undisturbed for 7 days before experiments. Animals were divided into 5 groups. For contractile in situ experiments, two groups of 5 mice received or a single dose of caged HwTxIV-Nvoc (5 mg/kg in 100 μL) or a similar intraperitoneally injection of 0.9% NaCl solution. For behavior actimeter experiments, three groups of 5 mice received a single intraperitoneally injection of HwTxIV analogue (0.5 mg/kg in 100 μL) or caged HwTxIV-Nvoc (5 mg/kg in 100 μL) or similar intraperitoneally injection of 0.9% NaCl solution. All procedures were conducted in conformity with European rules for animal experimentation (French Ethical Committee APAFIS #8186-2016121315485337, Jan. 10, 2017 for EDL contractile in situ experiments, APAFIS #1765-2016121315485337, Jan. 10, 2017 for motor behavior experiments with native toxin).

Electrophysiological Recordings from Neocortical Brain Slices

Neocortical coronal slices (350 μm thick) were prepared and maintained using previously described procedures adapted from other preparations (Jaafari, N. & Canepari, M. J Physiol 594, 967-983 (2016); Jaafari, N., De Waard, M. & Canepari, M. Biophys J 107, 1280-1288, (2014); Ait Ouares, K. & Canepari, M. J Neurosci 40, 1795-1809, (2020); Ait Ouares, K., et al. J Neurosci 39, 1969-1981 (2019)). Briefly, slices were incubated in extracellular solution at 37° C. for 45 min and then maintained at room temperature before use. The extracellular solution contained (in mM): 125 NaCl, 26 NaHCO₃, 1 MgSO₄, 3 KCl, 1 NaH₂PO₄, 2 CaCl₂) and 20 glucose, bubbled with 95% O₂ and 5% CO₂. The intracellular solution contained (in mM): 125 KMeSO₄, 5 KCl, 8 MgSO₄, 5 Na₂-ATP, 0.3 Tris-GTP, 12 Tris-Phosphocreatine, 20 HEPES, adjusted to pH 7.35 with KOH. Layer-5 (L5) pyramidal neurons from the somatosensory cortex were selected and patched in a whole cell configuration. Somatic action potentials (APs) were elicited by injecting current pulses of 3-5 ms duration and of 1-2 nA amplitude. In Na⁺ imaging experiments, after photo-release of the toxin, the current intensity was increased to 5-10 nA in order to depolarize the cell to the same Vₘ corresponding to the AP peak in control condition. The measured Vₘ was corrected for the junction and the bridge potentials. The caged toxin was dissolved in the extracellular solution at 2.5 μM concentration and locally delivered either using a SmartSquirt micro-perfusion system (WPI, Hitchin, UK) with a tip of 250 μm diameter, or by simple pressure ejection with a tip of ˜10 μm. Photolysis of the caged compound was performed either on a spot of ˜100 μm diameter and using the light of a 365 nm LED (˜2 mW of power) controlled by an OptoLED (Cairn Research, Faversham, UK), or on a spot of ˜40 μm using a 300 mW/405 nm diode laser (Cairn Research). The protocol at 365 nm consisted of 1-3 pulses of 100-300 ms duration and 1 s interval, or by a single pulse of 500 ms duration. The protocol at 405 nm consisted of 2 pulses of 500 ms duration and 5 s interval. In the case of simultaneous fluorescence recordings and UV stimulation, uncaging was optically combined to imaging by coupling the two excitation wavelengths similarly to what was done for an equivalent application (Vogt, K. E., et al. PLoS One 6, e24911, (2011)).

### Na⁺ Imaging

Optical measurements of [Na⁺] from the AIS were obtained as previously described (Filipis, L. & Canepari, M. J Physiol, (2020)). Briefly, neurons were loaded with 500 μM of the Na⁺ indicator ING-2 (IonBiosciences, San Marcos, TX, USA) for 20-30 min after establishing the whole-cell configuration. Fluorescence was excited using a 520 mW line of a LaserBank (Cairn Research) band-pass filtered at 517±10 nm, directed to the preparation using a 538 nm long-pass dichroic mirror. Emitted fluorescence was band-pass filtered at 559±17 nm before being recorded with a DaVinci 2K CMOS camera (SciMeasure, Decatur, GA) at 10 kHz with a pixel resolution of 30×128. Optical data, obtained by averaging 4 trials under the same condition, were corrected for bleaching using a trial without current injection. The fractional change of Na⁺ fluorescence was expressed in terms of intracellular Na⁺ concentration change (Δ[Na⁺]) using the previously published calibration for which 1% corresponds to 0.175 mM (Filipis, L. & Canepari, M. J Physiol, (2020)).

### Actimeter

Mice were randomly assigned to 3 groups according to toxin treatment as described above. The motor behavior was examined with an open field actimeter. For this analysis, mice were individually placed in an automated photocell activity chamber (Letica model LE 8811, Bioseb, France) which consists of a plexiglass chamber (20 cm×24 cm×14 cm) surrounded by two rows of infrared photobeams. The first row of sensors was raised at a height of 2 cm for measuring horizontal activity and the second row placed above the animal for vertical activity. Just after treatment administration via intraperitoneally injection, the spontaneous motor activity was measured for 10 min using a movement analysis system (Bioseb, France), which dissociates activity time (s), distance traveled (cm), total movements and rearing (numbers). All the experiments were realized in a dark room.

### Contractile Properties of Fast-Edl Muscle Using Sciatic Nerve Stimulation

EDL muscle force properties were analyzed by using in situ muscle contraction measurements as described previously (Carre-Pierrat, M. et al. Neuromuscul Disord 21, 313-327, (2011); Auda-Boucher, G. et al. Exp Cell Res 313, 997-1007, (2007)). Briefly, mice were anesthetized by intraperitoneally injection of xylazine/ketamine (10/100 mg/kg), and the adequacy of the anesthesia was monitored throughout the experiment. The skin was then carefully removed from the left leg, the sciatic nerve was carefully isolated and EDL muscle was dissected free with its blood supply intact. The foot and the tibia of the mice were fixed by two clamps, and the distal tendon of the EDL muscle was attached to a force transducer and positioned parallel to the tibia. Throughout the experimental procedure, the mice were kept on a heating pad to maintain normal body temperature, and the muscles were continuously perfused with Ringer solution. Stimulation electrodes were positioned at the level of the sciatic nerve, and connected to a pulse generator with stimulation characteristics of 0.2 ms duration, 6 V stimulation amplitude and 1 Hz frequency. The muscle was stretched, before stimulation voltage was applied to produce the most powerful twitch contractions, stimulation were maintained during all the experiments. Twitch parameters were measured prior to UV illumination, after 5 and 10 minutes of UV illumination (365 nm, 50 mW/cm²) and 5 and 10 minutes after UV illumination. At the end of the experiments, EDL muscles were rapidly dissected and weighted. Twitch forces were normalized in grams per milligram of fresh EDL muscles. All force data are expressed as percentage of the initial force, i.e. before illumination. All the experiments were realized in a dark room. Data were collected and stored for analysis with Chart v4.2.3 (PowerLab 4/25 ADInstrument, PHYMEP France).

### Statistics and Data Analysis

Values are represented as mean±SEM. Significance of illumination tests on automated patch-clamp was tested by performing paired 1-way ANOVA and Bonferroni's multiple and significance of in vivo experiments was tested by performing Friedman test with Dunn's multiple comparisons test. A p-value lower than 0.05 was considered significant. Significance of pharmacological tests on electrophysiological and optical recordings in brain slices was tested by performing the paired t-test with 0.01 as probability threshold value.

### RESULTS

### Design, Chemical Synthesis and Structure of a Caged HwTxIV Analogue

To demonstrate the applicability of modifying a venom peptide into a photosensitive tool in spite of the structural complexity and presence of multiple disulfide bridges, the inventors synthetized a pure caged HwTxIV-Nvoc compound based on a highly effective analogue of huwentoxin-IV (HwTxIVG¹G⁴K³⁶) (FIG. 1a). HwTxIV was first identified as a Na_{V}1.7 inhibitor but shown recently to target also Na_{V}1.6 with a lower affinity (Goncalves, T. C. et al. Neuropharmacology 133, 404-414, (2018)). The inventors first aimed to develop a HwTxIV analogue with convergent blocking potential for Na_{V}1.1, Na_{V}1.2 and Na_{V}1.6 channels. Based on earlier structure-function analyses using HwTxIV single amino acid mutations (Deng, M. et al. Toxicon: official journal of the International Society on Toxinology 71, 57-65, (2013); Agwa, A. J. et al. Biochim Biophys Acta Biomembr 1859, 835-844, (2017); Agwa, A. J. et al. J Biol Chem 295, 5067-5080, (2020)) and the recently resolved 3D structure of HwTxIV-Na_{V}1.7 complex (Shen, H., et al. Science 363, 1303-1308, (2019); Peng, K., et al. J Biol Chem 277, 47564-47571, (2002)), several residues were targeted to produce a more general Naᵥ channel inhibitor. By synthesizing multiple analogues and validating their functionality on Na_{V}1.1, Na_{V}1.2 and Na_{V}1.6 channels, the inventors determined that the HwTxIV analog (HwTxG¹G⁴K³⁶) was the most potent for the present study (Na_{V}1.1: 18.9±1.2 nM, n=3-10 for HwTxIV analog versus 50.7±1.2 nM, n=3-6 for HwTxIV; Na_{V}1.2: 11.9±1.1 nM, n=4-9 for HwTxIV analog versus 8.3±1.2 nM, n=6-11 for HwTxIV; Na_{V}1.6: 19.5±1.3 nM, n=6-10 for HwTxIV analog versus 154.3±1.2 nM, n=3-7 for HwTxIV; FIG. 1b). By mutating the K³² into N³² (IC₅₀>1 μM, n=4-7 for HwTxIV-N³² on Na_{V}1.7 versus IC₅₀=9.9 nM±0.9 nM, n=5-6 for HwTxIV on Na_{V}1.7), the inventors confirmed that the K³² residue is an ideal amino acid residue for chemical modifications aimed at reducing HwTxIV potency (FIG. 1c). Before proceeding with the chemical synthesis of caged HwTxIV-Nvoc analog, the inventors noted that K³² interacts with E⁸¹¹ of the S3 helix of VSD2-NavAb (pdb code 6N4R) and, if the binding of caged HwTxIV-Nvoc had to occur similarly to the non-caged HwTxIV, the presence of the Nvoc protecting group would introduce steric clashes. Caged HwTxIV-Nvoc was assembled stepwise using Fmoc-based SPPS on a 2-chlorotrityl-polystyrene resin, preloaded with a rink amide linker. The crude peptide was obtained in quantitative yield and of good quality to be directly oxidized. The presence of the Nvoc protecting group on K³² which is located next to the sixth Cys residue within the HwTxIV sequence can compromise the proper assembly of the three disulfide bridges within an ICK fold. The folding process was followed by analytical RP-HPLC and showed completion after 16 hours with the disappearance of the unfolded peptide and formation of one main oxidation peak occurring with a slight left shift on the elution profile. Finally, the pH of the reaction mixture was adjusted to 3 and the oxidized peptide was purified to homogeneity by preparative RP-HPLC. Two successive purifications, first using a C18 (5 μm, 130 Å) Waters CSH column, second using a C18 (10 μm, 100 Å) Phenomenex Luna stationary phase (eluent system H₂O/MeCN+0.1% TFA), were needed to complete production of the pure caged HwTxIV-Nvoc analog with a reasonably good global yield of 8.2% including SPPS, folding and purifications. The peptide was obtained with a measured exact mass of 722.1785 [M+6H]⁶⁺ by LC-ESI QTOF which is consistent with the theoretical mass of 722.1746. The proper mass indicates that the Nvoc protecting group was not removed by the TFA cleavage treatment of the synthetic crude unfolded peptide or during oxidative folding. As attested by the dispersion of the resonances, the non-caged HwTxIV is well structured. As expected, the presence of the aromatic Nvoc group on K³² for caged HwTxIV disturbs the chemical environments of a series of residues on the side of the molecule where K³² is located without affecting the global fold of the peptide. The presence of the grafted Nvoc group generates a doubling of certain peaks, possibly indicating different orientations of the Nvoc protecting group. Caged HwTxIV-Nvoc showed excellent dark stability in solution for over 1 week.

### Caged HwTxIV Analogue Drastically Reduces the Potency of the Peptide for Naᵥ Channels

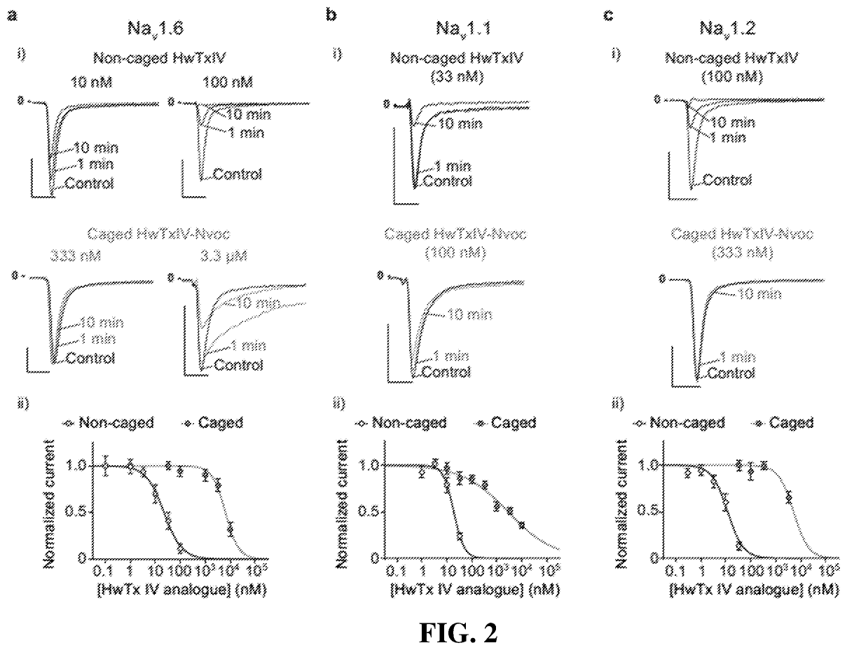

Next the inventors validated functionally that grafting the Nvoc group on HwTxIV analogue causes steric clashes important enough to reduce the Na_{V} channel blocking efficacy of HwTxIV analogue. Compared to non-caged HwTxIV, the caged toxin induces a 292-fold increase of the IC₅₀ value on Na_{V}1.6 (FIG. 2a) and even greater shifts are observed on Na_{V}1.1 and Na_{V}1.2 (FIG. 2b,c). Concentrations above 1 μM are needed for caged HwTxIV-Nvoc to start exhibiting inhibition of all Naᵥ channel subtypes tested. Remarkably, the toxin also slows fast inactivation of Na_{V}1.6 (FIG. 2a). The inventors interpret this effect as due to the existence of a low affinity binding locus on domain IV voltage sensor that is now revealed by the absence of a high affinity block due to Nvoc presence. To test this hypothesis, the inventors transplanted the S3-S4 motif from each of the four voltage-sensor domains (VSDI-IV) of Na_{V}1.6 into the homotetrameric K_{V}2.1 channel according to previously described boundaries (Bosmans, F., Martin-Eauclaire, M. F. & Swartz, K. J. Nature 456, 202-208, (2008); Bosmans, F., et al. J. Gen. Physiol. 138, 59-72, (2011); Osteen, J. D. et al. Nature 534, 494-499 (2016)). The transferred region in all of the functional chimeras contains the crucial basic residues that contribute to gating charge movement in K_{V} channels (Aggarwal, S. K. & MacKinnon, R. Neuron 16, 1169-1177 (1996); Seoh, S. A., et al. Neuron 16, 1159-1167 (1996); Ahern, C. A., et al. J Gen Physiol 147, 1-24 (2016)). Examination of conductance-voltage (G-V) relationships for the Na_{V}1.6/K_{V}2.1 chimeras revealed that each of the four voltage-sensor motifs has a distinct effect on the gating properties of K_{V}2.1. The inventors next examined the effect of caged HwTxIV-Nvoc on the Na_{V}1.6/K_{V}2.1 VSD chimeras. 5 μM toxin did not alter the G-V relationship of the VSDI and VSDIII chimera, whereas VSDII and VSDIV were clearly inhibited. Caged HwTxIV-Nvoc analogue binding to VSDII typically results in Naᵥ channel inhibition, whereas VSDIV is involved in channel fast inactivation although a role in channel opening may also be possible. Thus, the inventors conclude that, above 1 μM, HwTxIV-Nvoc influences Na_{V}1.6 gating primarily by interacting with the S3-S4 motif in VSDII and VSDIV.

### UV-Dependent Uncaging of HwTxIV-Nvoc Restores a Fully Functional HwTxIV Analogue

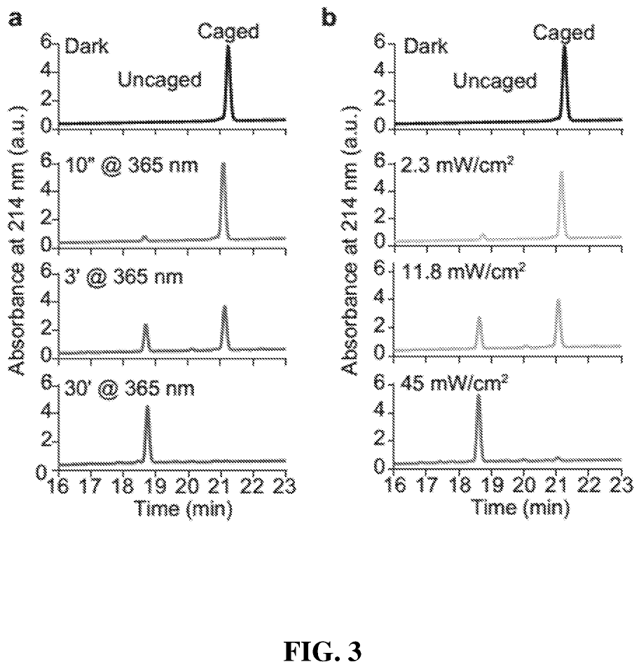

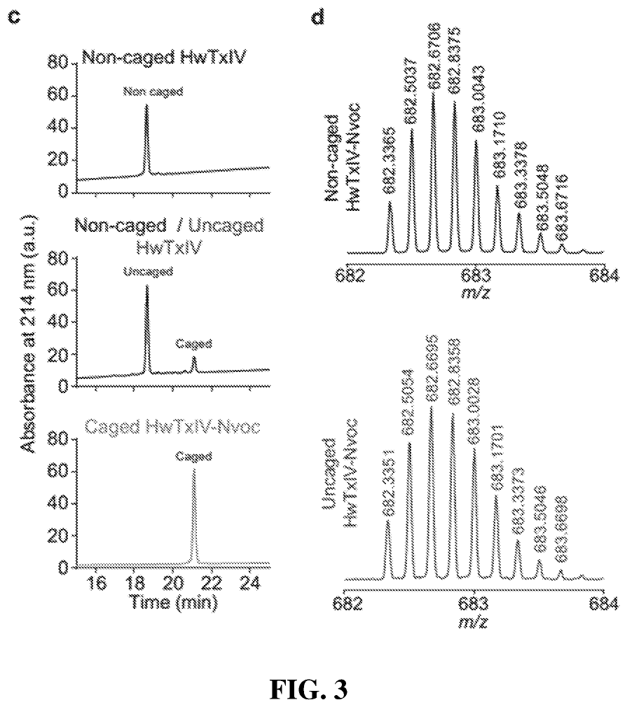

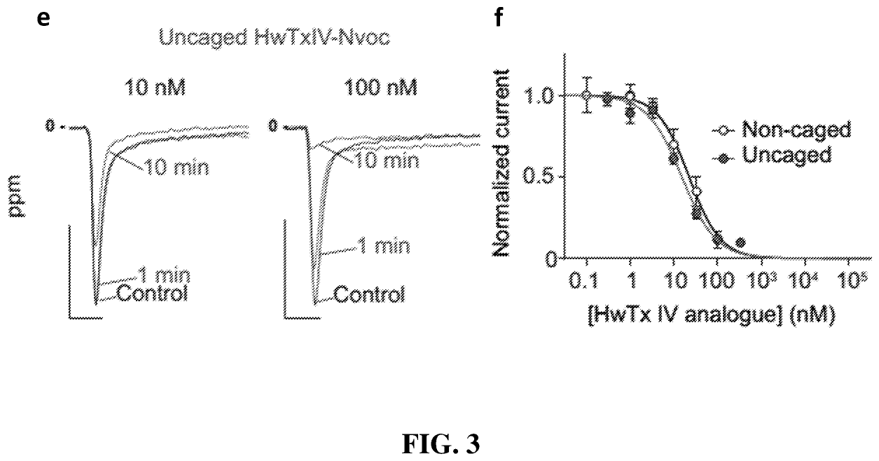

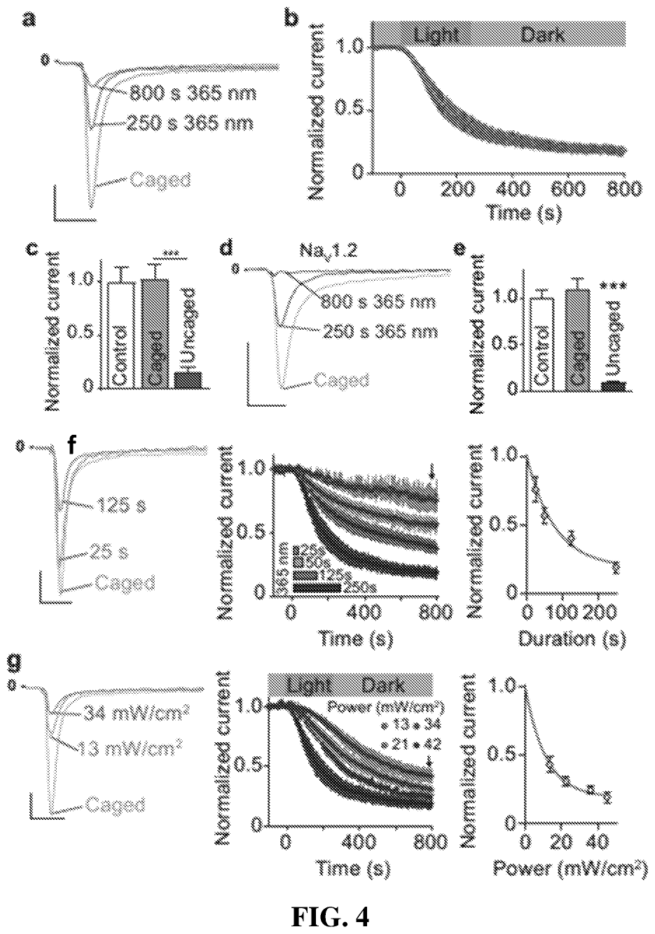

UV illumination at 365 nm of caged HwTxIV-Nvoc fully produced the uncaged HwTxIV analogue with an uncaging half-time of 3.6 min (45 mW/cm²) and half-power of 11.8 mW/cm² (FIG. 3a,b). HwTxIV analogue released from uncaging of HwTxIV-Nvoc has the same elution time by RP-HPLC and molecular weight as synthetic non-caged HwTxIV analogue (FIG. 3c,d). After illumination, the NMR spectra of uncaged HwTxIV-Nvoc and non-caged HwTxIV are perfectly superimposable indicating that illumination restores the predicted non-caged peptide. Also, photolysis of caged HwTxIV-Nvoc occurred from to λ=365 nm up to 405 nm but was negligible at wavelengths in the 435-740 nm range (10 min, power>18 mW/cm²) which offers the possibility to use the present technology in combination with fluorophores excited above 405 nm for additional monitoring techniques. Complete uncaging of HwTxIV analogue leads to inhibitory properties on Na_{V}1.6 that are identical to non-caged toxin, confirming structure preservation upon uncaging, but also indicating that the addition of the Nvoc protecting group did not lead to an abnormal disulfide bridging during peptide synthesis (FIG. 3 e,f). As expected from the various concentration-response curves measured so far, illumination of 100 nM caged HwTxIV-Nvoc, that is by itself inactive at this concentration, leads to a significant inhibition of Na_{V}1.6 currents (FIG. 4a-c). As expected of course, ending the illumination period does not restore Naᵥ1.6 current levels, as Nvoc deprotection is irreversible (FIG. 4b). Similar light-induced block of Naᵥ currents were observed with Na_{V}1.2 (FIG. 4d,e). The inventors also demonstrated that the extent of Na_{V}1.6 inhibition increases as a function of illumination time and power which is expected by the progressively larger quantities of uncaging occurring with illumination (FIG. 4f,g). As negative control, the inventors show that illumination alone (365 nm, 250 s, 45 mW/cm²) has no impact on Na⁺ current amplitude. Moreover, the Nvoc photolysis by-product resulting from uncaging 100 nM BeKm1-Nvoc toxin, used as a negative control since BeKm-1 does not act on Na_{V} channels, is inert on Na_{V}1.6.

### Generalisation of Caging Approach for Toxins Modulating Ion Channels

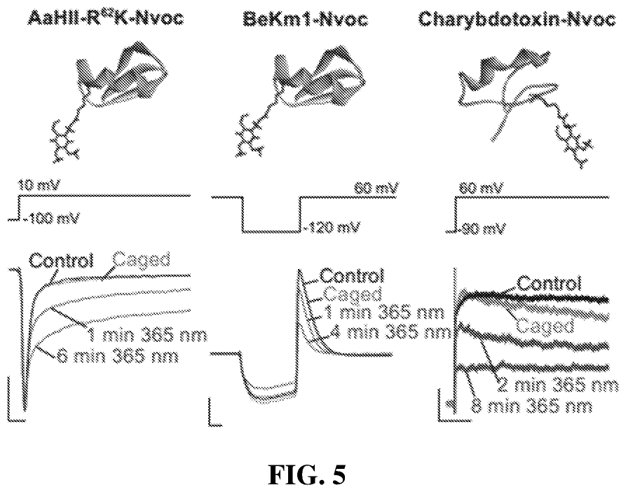

Similar inhibitory toxins displaying an important amine function on a key residue for pharmacology such as BeKm1 (hERG blocker) and charybdotoxin (K_{V}1.2 blocker) were also caged with a Nvoc protecting group. In addition, a Nvoc-grafted analogue of AaHII (a Na_{V} channel activator) was also produced wherein Arg⁶², another essential residue for function, was replaced by Lys⁶² to enlarge the applicability of the technique. Those toxins present similar uncaging efficacies, indicating that cage removal is largely toxin amino acid sequence- and conformation-independent suggesting a universal efficacy of this caging/uncaging approach for toxins. As expected for an appropriate positioning of Nvoc on the toxin sequence, uncaging by illumination at 365 nm of AaHIIR⁶²K-Nvoc, BeKm1-Nvoc or charybdotoxin-Nvoc lead to UV-dependent effects on Na_{V}1.2 (activation), hERG (block) and K_{V}1.2 (block) currents (FIG. 5), respectively, indicating that this approach can be generalized to toxin activators and pore blockers for the control of a wide diversity of ion channels.

### Spatio-Temporal Control of HwTxIV Analogue Activity in Brain Slices

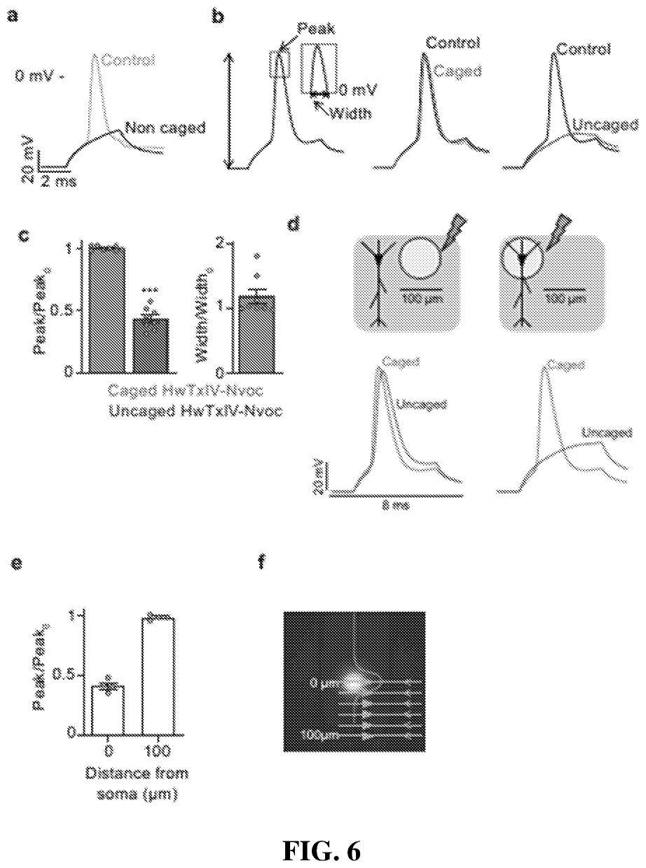

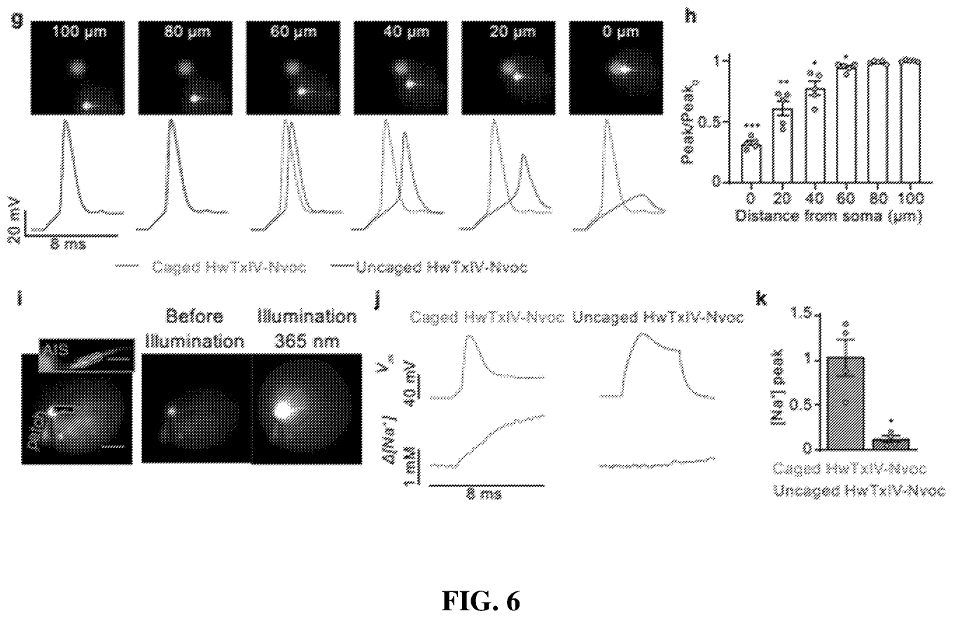

To expand the present approach to biological systems, the inventors assessed the use of HwTxIV-Nvoc in mouse brain slices during optical measurements of Na⁺ influx. The inventors first determined that action potentials (APs) recorded in neocortical layer-5 (L5) pyramidal neurons were inhibited by local application of 500 nM non-caged HwTxIV from the surface of the brain slice near the cell body (FIG. 6a). Similar experiments conducted with the caged HwTxIV-Nvoc analogue show that AP shape is unaltered (FIG. 6b,c). As expected, photolysis of HwTxIV-Nvoc leads to a significant decrease of the maximal membrane potential (Vₘ) 1 min after illumination (FIG. 6b,c). Next, spatial selectivity was first examined using a ˜100 μm diameter 365 nm UV LED spot. In this configuration, the somatic AP was not affected by illuminating a spot centered ˜100 μm away from the soma, whereas direct illumination of the soma inhibited the somatic AP (FIG. 6d,e). The inventors next used a more precise ˜40 μm diameter illumination spot to define the spatial conditions for toxin uncaging/AP blocking by positioning the spot at varying distances from the soma (FIG. 6f,g). The somatic AP was not affected when the cell was >80 μm from the spot center, whereas it was partially inhibited at distances between 60 and 20 μm from the spot center (FIG. 6h). These results provide information about the spatial resolution that can be attained by uncaging of HwTxIV-Nvoc inside the area of photolysis. The inventors then recorded Na⁺ influx via Naᵥ channels, associated with an AP, to unambiguously assess the effect of the uncaged toxin on the channels expressed in the axon initial segment (AIS) (FIG. 6i) using an ultrafast Na⁺ imaging approach. Illumination of caged HwTxIV-Nvoc in the soma (500 ms, 2 mW) fully prevented Na⁺ influx in the AIS, even at the same depolarized Vₘ of the AP (FIG. 6j,k). This result demonstrates that photo-release of HwTxIV analog blocks Na_{V} channels and AP propagation even at positive Vₘ. Altogether, these results provide proof-of-principle that controlled photolysis of caged HwTxIV-Nvoc can be used to precisely target AP initiation and Na⁺ influx in the AIS in brain slices.

### In Vivo Spatio-Temporal Control of Caged Toxins

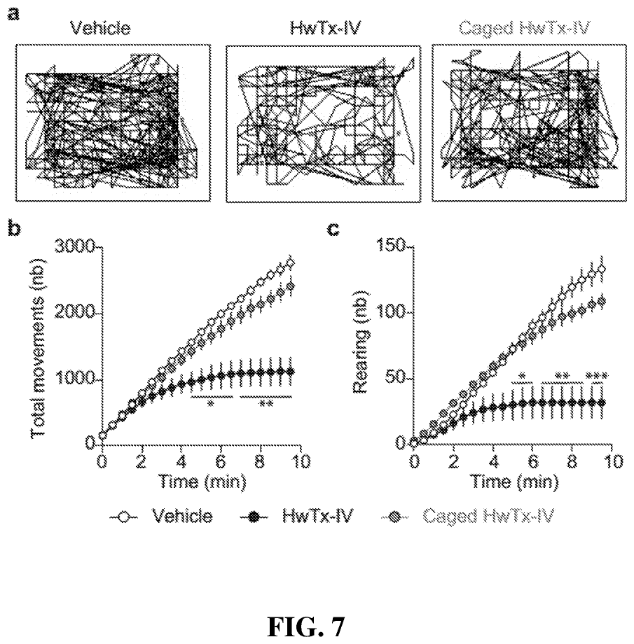

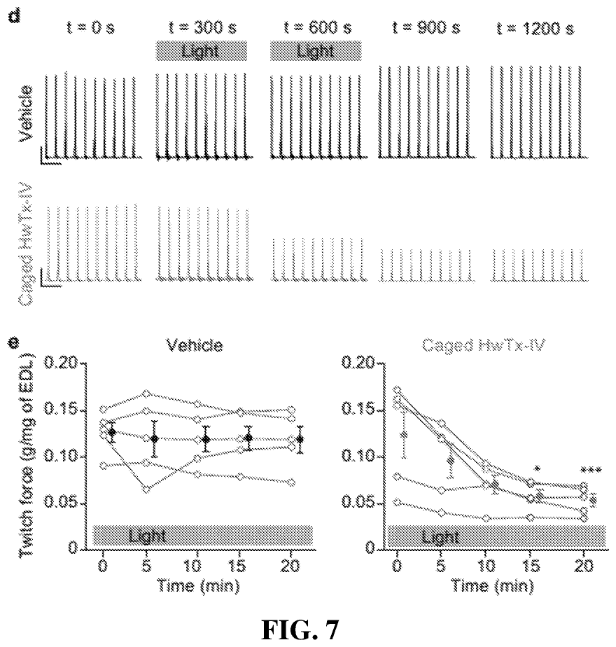

In order to evaluate the functional in vivo effects of the present approach, the inventors first validated that injection of caged HwTxIV-Nvoc has negligible effects on mice activity contrary to HwTxIV analogue which drastically reduce motor behaviour (FIG. 7a-c). Using in situ EDL muscle contraction preparation in anesthetized mice, the inventors validated that 365 nm illumination has no effects on EDL twitches and demonstrated that illumination of caged HwTxIV-Nvoc (365 nm, 50 mW/cm²) on neuromuscular junction significantly reduce contractile force (FIG. 7d-e). This result demonstrates, for the first time, in vivo activation of caged toxins using UV light in superficial tissues. By using a zebrafish larvae model, the inventors confirmed efficacy of this approach using caged AaHII-Nvoc toxin. Indeed, illumination induce paralysis in all larvaes previously injected with caged AaHII-Nvoc as observed with injection of non-caged AaHII toxin.

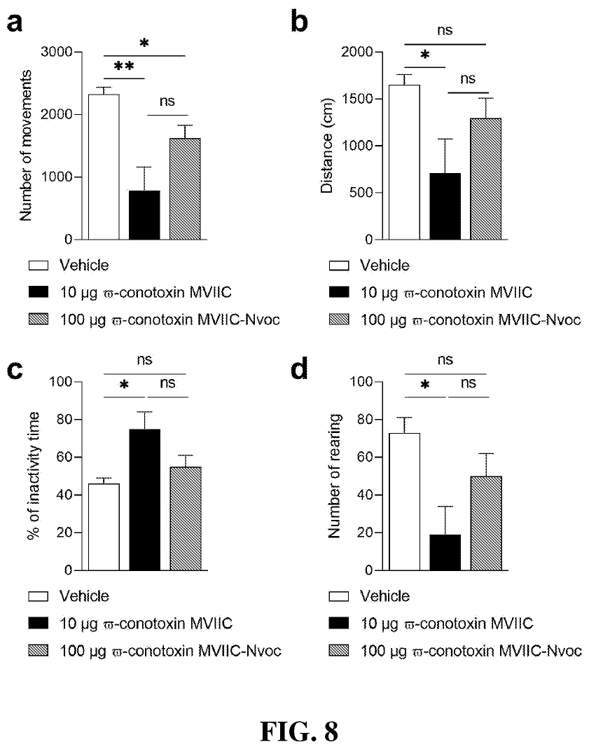

The inventors also evaluated the in vivo properties of non-caged and caged M-conotoxin MVIIC peptides (SEQ ID NO: 30) and showed that injection of caged M-conotoxin MVIIC has negligible effects on mice activity (FIGS. 8a-d).

Using in situ EDL muscle contraction preparation in anesthetized mice, the inventors validated that 365 nm illumination has no effects on EDL twitches and demonstrated that illumination of caged omega-contoxine-Nvoc (MVIIC-Nvoc)(365 nm, 50 mW/cm²) on neuromuscular junction significantly reduce contractile force (FIG. 8a-e). This result demonstrates, for the first time, in vivo activation of caged toxins using UV light in superficial tissues.

Caging of Jingzhaotoxin V (JzTxV, SEQ ID NO: 42) Drastically Reduces Affinity of JzTxV for hNaV1.4 Channels

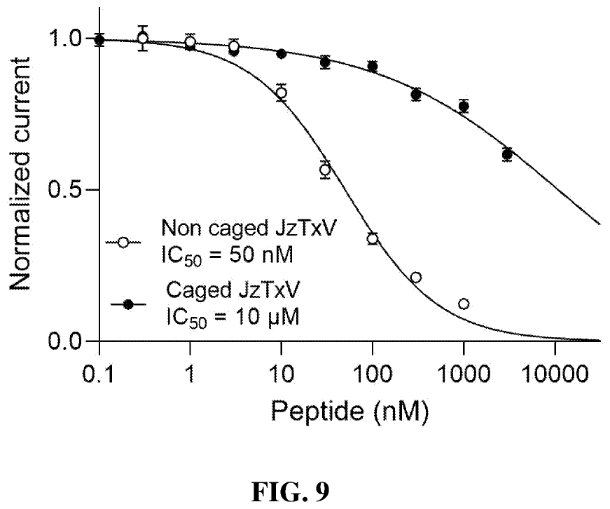

As shown in FIG. 9, caging of JzTxV drastically reduces affinity of JxTxV for hNaV1.4 channels (FIG. 9).

### DISCUSSION

In summary, the inventors report the development and application of a new, robust, generalizable and in vivo compatible strategy for producing photoactivatable toxins modulating voltage-gated ion channels and cell excitability. By using caged HwTxIV-Nvoc, the inventors demonstrate that caged compounds can be activated by wavelengths <435 nm and is therefore compatible with dyes used for optical imaging or fluorescent compounds to monitor voltage-dependent structural changes in ion channels. It is worth noting that this approach allows a spatial and temporal control of voltage-gated ion channel function. The present findings provide new opportunities for gaining insights into the functional investigation of ion channels in physiological or pathological processes such as sensory perception disorders, muscle and brain channelopathies or cardiac muscle diseases. The utility of classical pharmacology in vivo is limited, because local drug delivery is slow, imprecise, and hardly compatible with electrophysiology. In contrast, photopharmacology is able to mimic the timing, amplitude and spread of naturally occurring modulatory signals. By offering a real-time control of voltage-gated ion channels complexes and their functionalities in precisely defined regions that are poorly accessible to electrophysiological or pharmacological manipulations, photoactivatable toxins opened a new window for the understanding of physiology of ion channels. Similar approaches have been previously reported for ligand-gated ion channels (Noguchi, J. et al. J Physiol 589, 2447-2457, (2011)) and an elegant approach using coumarin-derived protected group grafted on saxitoxin targeting sodium channels has been recently published (Elleman, A. V. et al., Nature communications 12, 4171, (2021)). Despite very quick activation, this approach has been used on a non peptidic toxin and proof of in vivo efficacy has not been demonstrated yet. Given the number of peptides being isolated from venoms that target membrane proteins, the use of these photosensitive groups will first enable the development of a large number of new photoactivable toxins to better understand the functional heterogeneity of ion channels and next, by establishing causal relationships between a protein activity and a cellular or physiological output, photoactivatable toxins hold strong potential for identifying new therapeutic targets in many ion channels related diseases.

1-14. (canceled)

15. A method for treating an ion channel-related disease comprising administering a therapeutically efficient amount of a photoactivatable ion channel modulator, wherein said photoactivatable ion channel modulator is a disulphide-rich venom peptide comprising a photolabile protecting group.

16. The method of claim 15 wherein said photolabile protecting group is selected from the group consisting of: a nitrobenzyl-based photolabile protecting group, a carbonyl-based photolabile protecting group and a benzyl-based photolabile protecting group.

17. The method of claim 15 wherein said photolabile protecting group is 4,5-dimethoxy-2-nitrobenzyl (NVOC).

18. The method of claim 15 wherein a lysine, a tyrosine, a serine, a glycine or a cysteine of said disulphide-rich venom peptide is bound to said photolabile protecting group.

19. The method of claim 15 wherein the photoactivatable ion channel modulator is a photoactivatable voltage-gated sodium channel inhibitor.

20. The method of claim 15 wherein said photoactivatable ion channel modulator is Huwentoxin-IV comprising or consisting of an amino acid sequence selected from SEQ ID NO: 2 to 8 or a functional variant thereof and wherein lysine at position 32 is bound to the photolabile protecting group.

21. The method of claim 15 wherein the photoactivatable ion channel modulator is a photoactivatable voltage-gated sodium channel inhibitor and wherein said ion channel-related disease is a condition caused by abnormal cell excitability selected from the group consisting of: epilepsy, Dravet syndrome, convulsion, cardiac arrythmia, pain, erythromelalgia, lumbosacral radiculopathy and trigeminal neuralgia.

22. The method of claim 15 wherein said photoactivatable ion channel modulator is a photoactivatable voltage-gated ion channel activator and wherein said ion channel-related disease is a neuromuscular junction disorder.

23. The method of claim 15 wherein said photoactivatable ion channel modulator is a photoactivatable voltage-gated ion channel activator and wherein said ion channel-related disease is selected from the group consisting of: myasthenia gravis, an autoimmune neuromyotonia, a Lambert-Eaton syndrome, and a congenital and familial neuromuscular disorder.

24. The method of claim 15 wherein said photoactivatable ion channel modulator is a photoactivatable voltage-gated ion channel activator and wherein said ion channel-related disease is congenital myasthenia gravis syndrome.

25. A method for modulating activity of ion channel of a cell in a tissue, comprising administering a photoactivatable ion channel modulator to the tissue, wherein said photoactivatable ion channel modulator is a disulphide-rich venom peptide comprising a photolabile protecting group and wherein said disulphide-rich venom peptide is activated by irradiating said tissue at an appropriate wavelength of light.

26. The method of claim 25 wherein said photolabile protecting group is a nitrobenzyl-based photolabile protecting group and said disulphide-rich venom peptide is activated by irradiating said tissue at a wavelength above 340 nm.

27. The method of claim 25, wherein the method is employed to reduce a soft-tissue feature.

28. The method of claim 25 wherein said photoactivatable ion channel modulator is Huwentoxin-TV comprising or consisting of an amino acid sequence selected from SEQ ID NO: 2 to 8 or a functional variant thereof and wherein lysine at position 32 is bound to the photolabile protecting group.

29. A disulphide-rich venom peptide comprising a photolabile protecting group wherein said disulphide-rich venom peptide is
a Huwentoxin-IV comprising or consisting of an amino sequence selected from SEQ ID NO: 2 to 8 or a functional variant thereof wherein a lysine at position 32 is bound to a photolabile protecting group,
a Charybdotoxin comprising or consisting of SEQ ID NO: 10 or a functional variant thereof wherein a lysine at position 27, an asparagine at position 30 or tyrosine at position 36 is bound to a photolabile protecting group, or
an AahII comprising or consisting of SEQ ID NO: 12 or a functional variant thereof wherein a residue at position 62 is bound to a photolabile protecting group.

30. The disulphide-rich venom peptide of claim 29 wherein said disulphide-rich venom peptide is the Charybdotoxin comprising or consisting of SEQ ID NO: 10 or a functional variant thereof, wherein the lysine at position 27, the asparagine at position 30 or the tyrosine at position 36 is bound to the photolabile protecting group.

31. The disulphide-rich venom peptide of claim 29 wherein said disulphide-rich venom peptide is the AahII comprising or consisting of SEQ ID NO: 12 or a functional variant thereof, wherein an arginine at position 62 is replaced by a lysine which is bound to said photolabile protecting group.

32. The disulphide-rich venom peptide according to claim 29 wherein said photolabile protecting group is selected from the group consisting of: a nitrobenzyl-based photolabile protecting group, a carbonyl-based photolabile protecting group and a benzyl-based photolabile protecting group.

33. The disulphide-rich venom peptide according to claim 29 wherein said photolabile protecting group is 4,5-Dimethoxy-2-nitrobenzyl (NVOC).


SEQUENCE LISTING (43 sequences)

SEQ ID NO: 1 - protein, 89 aa, Haplopelma schmidti
MVNMKASMFLALAGLVLLFVVCYASESEEKEFSNELLSSVLAVDDNSKGEERECLEIFKACNPSNDQCCKSSKLVCSRKTRWCKYQIGK

SEQ ID NO: 2 - protein, 37 aa, Haplopelma schmidti
ECLEIFKACNPSNDQCCKSSKLVCSRKTRWCKYQIGK

SEQ ID NO: 3 - protein, 37 aa, synthetic construct
  REGION 1..37: note = Huwentoxin-IV variant
ECLGIFKACNPSNDQCCKSSKLVCSRKTRWCKYQIGK

SEQ ID NO: 4 - protein, 37 aa, synthetic construct
  REGION 1..37: note = Huwentoxin-IV variant
ECLKIFKACNPSNDQCCKSSKLVCSRKTRWCKYQIGK

SEQ ID NO: 5 - protein, 37 aa, synthetic construct
  REGION 1..37: note = Huwentoxin-IV variant
ECLRIFKACNPSNDQCCKSSKLVCSRKTRWCKYQIGK

SEQ ID NO: 6 - protein, 37 aa, synthetic construct
  REGION 1..37: note = Huwentoxin-IV variant
GCLGIFKACNPSNDQCCKSSKLVCSRKTRWCKYQIGK

SEQ ID NO: 7 - protein, 37 aa, synthetic construct
  REGION 1..37: note = Huwentoxin-IV variant
ECLKIFKACNPSNDQCCKSSKLVCSQKTRWCKYQIGK

SEQ ID NO: 8 - protein, 37 aa, synthetic construct
  REGION 1..37: note = Huwentoxin-IV variant
GCLGIFKACNPSNDQCCASSKLVCSRKTRWCKYQIGK

SEQ ID NO: 9 - protein, 58 aa, unidentified
MKILSVLLLALIICSIVGWSAQFTNVSCTTSKECWSVCQRLHNTSRGKCMNKKCRCYS

SEQ ID NO: 10 - protein, 37 aa, unidentified
QFTNVSCTTSKECWSVCQRLHNTSRGKCMNKKCRCYS

SEQ ID NO: 11 - protein, 85 aa, unidentified
MNYLVMISLALLFVTGVESVKDGYIVDDVNCTYFCGRNAYCNEECTKLKGESGYCQWASPYGNACYCYKLPDHVRTKGPGRCHGR

SEQ ID NO: 12 - protein, 66 aa, unidentified
VKDGYIVDDVNCTYFCGRNAYCNEECTKLKGESGYCQWASPYGNACYCYKLPDHVRTKGPGRCHGR

SEQ ID NO: 13 - protein, 35 aa, unidentified
ACREWLGGCSKDADCCAHLECRKKWPYHCVWDWTV

SEQ ID NO: 14 - protein, 80 aa, unidentified
MMNRLLVFLMLGAAFMLVVSAIDQDANEDINKRGVPCLCDSDGPSVRGNTLSGIIWLAGCPSGWHNCKKHGPTIGWCCKQ

SEQ ID NO: 15 - protein, 47 aa, unidentified
GVPCLCDSDGPSVRGNTLSGIIWLAGCPSGWHNCKKHGPTIGWCCKQ

SEQ ID NO: 16 - protein, 82 aa, unidentified
MKLTCVVIVAALLLTACQLITALDCGGTQKHRALRSTIKLSLLRQHRGWCGDPGATCGKLRLYCCSGFCDCYTKTCKDKSSA

SEQ ID NO: 17 - protein, 34 aa, unidentified
WCGDPGATCGKLRLYCCSGFCDCYTKTCKDKSSA

SEQ ID NO: 18 - protein, 64 aa, unidentified
IKDGYIVDDVNCTYFCGRNAYCNEECTKLKGESGYCQWASPYGNACYCYKLPDHVRTKGPGRCR

SEQ ID NO: 19 - protein, 34 aa, Paraphysa scrofa
DCLGFLWKCNPSNDKCCRPNLVCSRKDKWCKYQI

SEQ ID NO: 20 - protein, 86 aa, unidentified
MKASMFLALTGLALLFVVCYASESEEKEFSNELLSSVLAVDDNSKGEERECLGFGKGCNPSNDQCCKSSNLVCSRKHRWCKYEIGK

SEQ ID NO: 21 - protein, 35 aa, unidentified
ECLGFGKGCNPSNDQCCKSSNLVCSRKHRWCKYEI

SEQ ID NO: 22 - protein, 48 aa, unidentified
GGVPCLCDSDGPSVRGNTLSGIIWLAGCPSGWHNCKAHGPTIGWCCKQ

SEQ ID NO: 23 - protein, 32 aa, unidentified
RDCQEKWEYCIVPILGFVYCCPGLICGPFVCV

SEQ ID NO: 24 - protein, 48 aa, unidentified
GVACRCDSDGPTVRGDSLSGTLWLTGGCPSGWHNCRGSGPFIGYCCKK

SEQ ID NO: 25 - protein, 30 aa, unidentified
YCQKWMWTCDSERKCCEGMVCRLWCKKKLW

SEQ ID NO: 26 - protein, 33 aa, unidentified
DCLKFGWKCNPRNDKCCSGLKCGSNHNWCKLHI

SEQ ID NO: 27 - protein, 35 aa, unidentified
ECRYWLGGCSAGQTCCKHLVCSRRHGWCVWDGTFS

SEQ ID NO: 28 - protein, 33 aa, unidentified
DCGTIWHYCGTDQSECCEGWKCSRQLCKYVIDW

SEQ ID NO: 29 - protein, 48 aa, unidentified
KKKCIAKDYGRCKWGGTPCCRGRGCICSIMGTNCECKPRLIMEGLGLA

SEQ ID NO: 30 - protein, 29 aa, unidentified
TRCKGKGAPCRKTMYDCCSGSCGRRGKCG

SEQ ID NO: 31 - protein, 39 aa, synthetic construct
  REGION 1..39: note = Huwentoxin-XVI
  SITE 29: note = misc_feature - Xaa can be any naturally occurring amino acid
CIGEGVPCDENDPRCCSGLVCLKPTLHGXWYKSYYCYKK

SEQ ID NO: 32 - protein, 71 aa, unidentified
MKLTCVVIVAVLLLTACQLITADDSRGTQKHRALRSTTKLSTSTRCKGKGAKCSRLMYDCCTGSCRSGKCG

SEQ ID NO: 33 - protein, 25 aa, unidentified
CKGKGAKCSRLMYDCCTGSCRSGKC

SEQ ID NO: 34 - protein, 71 aa, unidentified
MKLTCMVIVAVLLLTACQLITADDSRGTQKHRTLRSKTKLSMSTRCKAAGKPCSRIAYNCCTGSCRSGKCG

SEQ ID NO: 35 - protein, 25 aa, unidentified
CKAAGKPCSRIAYNCCTGSCRSGKC

SEQ ID NO: 36 - protein, 41 aa, unidentified
GVDKAGCRYMFGGCSVNDDCCPRLGCHSLFSYCAWDLTFSD

SEQ ID NO: 37 - protein, 22 aa, unidentified
GGKPDLRPCHPPCHYIPRPKPR

SEQ ID NO: 38 - protein, 15 aa, unidentified
ECCNPACGRHYSCGK

SEQ ID NO: 39 - protein, 14 aa, unidentified
GRCCHPACGKNYSC

SEQ ID NO: 40 - protein, 85 aa, unidentified
MQTAYWVMVMMMVMWITAPLSEGGKPKLIIRGLVPNDLTPQRILRSLISGRTYGIYDAKPPFSCAGLRGGCVLPPNLRPKFKEGR

SEQ ID NO: 41 - protein, 32 aa, unidentified
TYGIYDAKPPFSCAGLRGGCVLPPNLRPKFKE

SEQ ID NO: 42 - protein, 29 aa, unidentified
YCQKWMWTCDSKRACCEGLRCKLWCRKII

SEQ ID NO: 43 - protein, 26 aa, unidentified
CKGKGAPCRKTMYDCCSGSCGRRGKC